(19) United States
(12) Patent Application Publication
(10) Pub. No.: US 2004/0110738 A1
(43) Pub. Date: Jun. 10, 2004

(54) Prophylactic treatment methods

(76) Inventors: Scott H. Gillis, Concord, MA (US); Paul Schechter, Dover, MA (US); Robert H. Demling, Natick, MA (US)

Correspondence Address: FISH & RICHARDSON PC, 225 FRANKLIN ST, BOSTON, MA, 02110, US

(21) Appl. No.: 10/690,710

(22) Filed: Oct. 22, 2003

Related U.S. Application Data
(60) Provisional application No. 60/420,167, filed on Oct. 22, 2002.

(51) Int. Cl. A61K 31/555; A61K 31/28; A61K 33/24

(52) U.S. Cl. 514/184; 424/617; 514/492

(57) ABSTRACT

Prophylactic treatment methods are disclosed. The methods can include contacting an object and/or an area of a subject with a metal-containing material to reduce the occurrence of a condition at the same area or a different area of the subject. The metal-containing material can be, for example, an antimicrobial material, an anti-biofilm metal-containing material, an antibacterial material, an anti-inflammatory material, an anti-fungal material, an anti-viral material, an anti-cancer material, a pro-apoptosis material, an anti-proliferative material, an MMP modulating material, an atomically disordered, crystalline material, and/or a nanocrystalline material. In certain embodiments, the metal-containing material is an atomically disordered, nanocrystalline silver-containing material.

### CROSS-REFERENCE TO RELATED APPLICATION

This application claims priority under 35 U.S.C. §119 to U.S. Provisional patent application Serial No. 60/420,167, filed Oct. 22, 2002, which is hereby incorporated by reference.

### INCORPORATION BY REFERENCE

The following U.S. patent applications are incorporated herein by reference: U.S. Ser. No. 10/277,673, filed Oct. 22, 2002; U.S. Ser. No. 10/277,356, filed Oct. 22, 2002; U.S. Ser. No. 10/277,298, filed Oct. 22, 2002; U.S. Ser. No. 10/277,362, filed Oct. 22, 2002; U.S. Ser. No. 10/277,358, filed Oct. 22, 2002; U.S. Ser. No. 10/277,320, filed Oct. 22, 2002; U.S. Ser. No. ______ [Attorney Docket: 14072-035001], filed Oct. 22, 2003; U.S. Ser. No. ______ [Attorney Docket: 14072-036001], filed Oct. 22, 2003; and U.S. Ser. No. ______ [Attorney Docket: 14072-037001], filed Oct. 22, 2003.

### TECHNICAL FIELD

The invention relates to prophylactic treatment methods.

### BACKGROUND

It is known to use certain chemical compounds to prophylactically treat a subject. Prophylactically treating a subject often involves contacting the subject with one or more chemical compounds that are effective in reducing the likelihood that the condition will occur in the subject.

### SUMMARY

The invention relates to prophylactic treatment methods.

In one aspect, the invention relates to a method of prophylactically treating a condition. The method includes contacting a first area of a subject with a metal-containing material to reduce the occurrence of the condition at a second area of the subject. The first and second areas of the subject can be the same area of the subject, or the first and second areas of the subject can be different areas of the subject.

In another aspect, the invention relates to a method of prophylactically treating a condition. The method includes contacting an object (e.g., a medical device, a mechanical mister, a spray bottle, a nebulizer, an oxygen tent, a dry powder inhaler, a needle, a needleless injector, a dressing, a solution dropper, a containers for a solution) with a metal-containing material to reduce the occurrence of the condition at an area of a subject. The object is intended to be contacted with the subject or a material in contact with the object is intended to be contacted with the subject.

Embodiments can include one or more of the following features.

In embodiments that include contacting first and second areas (which may the same or different areas) of the subject with the metal-containing material, the method can further include recognizing a possibility for occurrence of the condition at the second area of the subject, and, after recognizing the possibility for occurrence of the condition at the second area of the subject, selecting the first area of the subject for contact with the metal-containing material to reduce occurrence of the condition at the second area.

In embodiments that include contacting an object with the metal-containing material, the method can further include recognizing a possibility for occurrence of the condition at the area of the subject, and after, recognizing the possibility for occurrence of the condition at the area of the subject, selecting the object for contact with the metal-containing material to reduce occurrence of the condition at the area of the subject.

In embodiments that include contacting an object with the metal-containing material, the method can further include, after contacting the object with the metal-containing material, contacting the object with the subject. The object can contacted with the same area of the subject or a different area of the subject.

In embodiments that include contacting an object with the metal-containing material, the method can further include, after contacting the object with the metal containing material, transferring from the object to the subject the material intended to be transferred to the subject. The material transferred to the subject can be, for example, a therapeutic agent. The material can directly or indirectly transferred directly from the object to the subject.

The methods can include monitoring a subject after contacting the subject with the metal-containing material. For example, a subject can be monitored at relatively regular intervals (e.g., about once an hour, about once every eight hours, about once a day, about once a week, about two times a month, about three times a month, about four times a month).

The metal-containing material can be, for example, an alloy or a metal.

Examples of metal-containing materials include metal oxides, metal nitrides, metal borides, metal carbides, metal nitrates, metal hydroxides, metal carbonates, metal sulfides, metal sulfadiazines, metal halides, metal phosphides, metal silicates, metal acetates, metal lactates, metal citrates, metal myristates, metal sorbates, metal stearates, metal oleates, metal glutonates, metal adipates, alkali metal thiosulphates (e.g., sodium metal thiosulphate, potassium metal thiosulphate) and metal hydrides.

The metal-containing material can contain, for example, silver, gold, platinum and/or palladium.

The metal-containing material can be ionic.

The metal-containing material can be, for example, an atom, a molecule or a cluster.

The metal-containing material can be, for example, an antimicrobial material, an anti-biofilm material, an antibacterial material, an anti-inflammatory material, an antifungal material, an antiviral material, an anti-autoimmune material, an anti-cancer material, a pro-apoptosis material, anti-proliferative, and/or MMP modulating material.

The metal-containing material can be, for example, a nanocrystalline material.

The metal-containing material can be, for example, an atomically disordered, crystalline material.

The condition can be, for example, a bacterial condition, a microbial condition, an inflammatory condition, a fungal condition, a viral condition, an autoimmune condition, an idiopathic condition, a hyperproliferative condition, a noncancerous growth and/or a cancerous condition.

The condition can be, for example, a skin condition or an integument condition. Examples of skin conditions or integument conditions include bums, eczema (e.g., atopic eczema, acrodermatitis continua, contact allergic dermatitis, contact irritant dermatitis, dyshidrotic eczema, pompholyx, lichen simplex chronicus, nummular eczema, seborrheic dermatitis, stasis eczema), erythroderma, insect bites, mycosis fungoides, pyoderma gangrenosum, eythrema multiforme, rosacea, onychomycosis, acne (e.g., acne vulgaris, neonatal acne, infantile acne, pomade acne), psoriasis, Reiter's syndrome, pityriasis rubra pilaris, hyperpigmentation, vitiligo, scarring conditions, keloids, lichen planus, age-related skin disorders (e.g., wrinkles, cellulite) and hyperproliferative variants of the disorders of keratinization (e.g., actinic keratosis, senile keratosis).

The condition can be, for example, a respiratory condition. Examples of respiratory conditions include asthma, emphysema, bronchitis, pulmonary edema, acute respiratory distress syndrome, bronchopulmonary dysplasia, pulmonary fibrosis, pulmonary atelectasis, tuberculosis, pneumonia, sinusitis, allergic rhinitis, pharyngitis, mucositis, stomatitis, chronic obstructive pulmonary disease, bronchiectasis, lupus pneumonitis and cystic fibrosis.

The condition can be, for example, a musculo-skeletal condition. Examples of musculo-skeletal conditions include tendonitis, osteomyelitis, fibromyalgia, bursitis and arthritis.

The condition can be, for example, a circulatory condition. Examples of circulatory conditions include arteriosclerosis, lymphoma, septicemia, leukemia, ischemic vascular disease, lymphangitis and atherosclerosis.

The condition can be, for example, a cancerous condition. Examples of cancerous conditions include tumors and hematologic malignancies.

The condition can be, for example, a noncancerous growth.

The condition can be, for example, a mucosal condition or a serosal condition. Examples of mucosal or serosal conditions include pericarditis, Bowen's disease, stomatitis, prostatitis, sinusitis, allergic rhinitis, digestive disorders, peptic ulcers, esophageal ulcers, gastric ulcers, duodenal ulcers, espohagitis, gastritis, enteritis, enterogastric intestinal hemorrhage, toxic epidermal necrolysis syndrome, Stevens Johnson syndrome, cystic fibrosis, bronchitis, pneumonia (e.g., nosocomial pneumonia, ventilator-associated pneumonia), pharyngitis, common cold, ear infections, sore throat, sexually transmitted diseases (e.g., syphilis, gonorrhea, herpes, genital warts, HIV, chlamydia), inflammatory bowel disease, colitis, hemorrhoids, thrush, dental conditions, oral conditions, conjunctivitis, and periodontal conditions.

The method can be used to prophylactically induce apoptosis at the second area of the subject.

The method can be sued to prophylactically modulate matrix metalloproteinases at the second area of the subject.

The area of the subject at which the condition is susceptible to occur can be, for example, a portion of the skin, a nail, a portion of the respiratory system (e.g., a portion of the oral cavity, a portion of the nasal cavity, a portion of at least one lung), a portion of the musculo-skeletal system (e.g., a portion of a bone, a portion of a joint, a portion of a muscle, a portion of a tendon) a portion of the circulatory system (e.g., a portion of the heart, a portion of the lymphatic system, a portion of blood, a portion of a blood vessel), a portion of the gastrointestinal system (e.g., a portion of the oral cavity, a portion of the colon, a portion of the small intestine, a portion of the large intestine, a portion of the stomach, a portion of the esophagus), a portion of the sublingual area, or a portion of the subdermal area. The area of the subject at which the condition is susceptible to occur can be, for example, any area of the subject where there is a hyperplastic tissue, a tumor tissue, a noncancerous growth or a cancerous lesion.

The area of the subject contacted with the metal-containing material can be, for example, a portion of the skin, a portion of the respiratory system (e.g., a portion of the oral cavity, a portion of the nasal cavity, a portion of at least one lung), a portion of the musculo-skeletal system (e.g., a portion of a bone, a portion of a joint, a portion of a muscle, a portion of a tendon) a portion of the circulatory system (e.g., a portion of the heart, a portion of the lymphatic system, a portion of blood, a portion of a blood vessel), a portion of the gastrointestinal system (e.g., a portion of the oral cavity, a portion of the colon, a portion of the small intestine, a portion of the large intestine, a portion of the stomach, a portion of the esophagus), a portion of the sublingual area, or a portion of the subdermal area. The area of the subject contacted with the metal-containing material can be, for example, any area of the subject where there is a hyperplastic tissue, a tumor tissue and a cancerous lesion.

The subject can be, for example, a human or an animal.

The metal-containing material can be in a solution when contacted with the subject. The solution is injected (e.g., via a needleless injector, via a needle). The solution can contain at least about 0.001 weight percent of the metal-containing material. The solution can contain about 10 weight percent or less of the metal-containing material. The solution can include a solvent. In certain embodiments, the method can include forming the solution into an aerosol and inhaling the aerosol. In some embodiments, the method can include forming the solution into a spray and spraying onto or into the body.

The metal-containing material can be disposed in a pharmaceutically acceptable carrier when contacted with the subject. The composition can contain at least about 0.01 weight percent of the nanocrystalline metal-containing material. The composition can contain about 50 weight percent or less of the nanocrystalline metal-containing material. The pharmaceutically acceptable carrier can be, for example a cream, an ointment, a gel, a lotion, a paste, a foam or a liposome (e.g., in the form of a lozenge, a tape, a tablet, a suppository, a pill, or a capsule).

The metal-containing material can be in the form of a free standing powder when contacted with the subject. The free standing powder can be inhaled. In some embodiments, the free standing powder can be injected into the body. In certain embodiments, the free standing powder can be sprinkled onto a body part.

The metal-containing material can be, for example, in the form of a swab, a sponge, a coated tube (e.g., used for miringotomy), a foam, a liposome, a tape, a pill, a capsule, a tablet, a suppository or a lozenge when contacted with the subject.

The first area can be. a mucosal membrane (e.g., the subject's oral cavity and/or the subject's nasal cavity), and the second area can be the subject's lungs.

The condition can be, for example, nosocomial pneumonia or ventilator-associated pneumonia.

The second area of the subject can be substantially free of the condition when the first area of the subject is contacted with the metal-containing material.

The second area of the subject can have the condition when the first area of the subject is contacted with the metal-containing material.

The first area of the subject can be substantially free of the condition when the first area of the subject is contacted with the metal-containing material.

The first area of the subject can have the condition when the first area of the subject is contacted with the metal-containing material.

The metal-containing material can have a prophylactic ratio of about 0.95 or less for the condition.

In certain embodiments, the metal-containing material is in a form other than a dressing.

In some embodiments, the condition is not a bacterial condition.

In certain embodiments, the first area of the subject is not a portion of the subject's skin.

Other features and advantages of the methods will be apparent from the description and drawings, and from the claims.

### DESCRIPTION OF DRAWINGS

FIG. 1 is a schematic view of a deposition system.

### DETAILED DESCRIPTION

In general, the invention relates to prophylactic treatment methods. Typically, the methods include contacting an area of a subject with a metal-containing material (e.g., an antimicrobial, anti-biofilm, antibacterial, anti-inflammatory, antifungal, antiviral, anti-autoimmune, anti-cancer, pro-apoptosis, anti-proliferative, MMP modulating, atomically disordered, crystalline, and/or nanocrystalline silver-containing material) to reduce (e.g., prevent) the occurrence of a condition at the same area or a different area of the subject. Generally, when the area of the subject is contacted with the metal-containing material, the area of the subject that is prophylactically treated is substantially without the condition.

Without wishing to be bound by theory, in embodiments in which the area of the subject contacted with the material is the same as the area of the subject being prophylactically treated, it is believed that the metal-containing material reduces the occurrence of the condition at the area of the subject by reducing the presence at the area of the subject of one or more pathogens of the condition (e.g., one or more prions, parasites, fungi, viruses, inflammatory agents, cancer cells, allergens and/or bacteria).

Without wishing to be bound by theory, in embodiments in which the area contacted with the metal-containing compound is different from the area being prophylactically treated, it is believed that the observed therapeutic effect may be explained by one or more potential mechanisms. In one potential mechanism, it is believed that the metal-containing material reduces the occurrence of the condition at the prophylactically treated area of the subject by reducing the presence at the contacted area of the subject of one or more pathogens of the condition (e.g., one or more prions, parasites, fungi, viruses, inflammatory agents, cancer cells, allergens and/or bacteria) that can move from the first area of the subject to the second area of the subject. In another potential mechanism, it is believed that the metal-containing material causes the release of a mediator (e.g., a biological mediator) within the subject, and the mediator enters (or is formed in) the circulatory system of the subject so that the mediator circulates to the portion of the subject that is susceptible to the condition (the area of prophylactic treatment) where the mediator directly or indirectly provides the observed therapeutic effect (e.g., by reducing the presence of one or more pathogens of the condition). In a further potential mechanism, it is believed that the metal-containing material itself enters the circulatory system of the subject so that the material is circulated to the portion of the subject susceptible to the condition (the area of prophylactic treatment) where the material provides its therapeutic effect (either directly or indirectly). In an additional potential mechanism, it is believed that the metal-containing material can assist in maintaining a number and/or concentration of one or more pathogens (e.g., one or more prions, parasites, fungi, viruses, inflammatory agents, cancer cells, allergens and/or bacteria) of a condition below a level that can be dangerous to a subject (e.g., below a level corresponding to an infection). It is believed that combinations of potential mechanisms may result in the observed therapeutic effect of the metal-containing material.

As an example, a metal-containing material (e.g., an antimicrobial, anti-biofilm, antibacterial, anti-inflammatory, antifungal, antiviral, anti-autoimmune, anti-cancer, pro-apoptosis, anti-proliferative, and/or MMP modulating atomically disordered , nanocrystalline silver-containing material) can be contacted with the subject's nasal cavity and/or oral cavity to reduce the occurrence of pneumonia (e.g., nosocomial pneumonia, ventilator-associated pneumonia) in the lungs of the subject. The metal-containing material can be in the form of, for example, a solution, a mist, a swab, a sponge, a coated tube (e.g., used for miringotomy), a foam, a liposome, a tape, a pill, a capsule, a tablet, a suppository and/or a lozenge. Without wishing to be bound by theory, it is believed that this method reduces the occurrence of pneumonia in the subject by reducing the presence in the subject's nasal cavity and/or oral cavity of one or more pathogens of pneumonia (e.g., one or more prions, parasites, fungi, viruses, inflammatory agents, cancer cells, allergens and/or bacteria) that can move from the subject's nasal cavity and/or oral cavity to the subject's lungs and result in the occurrence of pneumonia.

As another example, a metal-containing material (e.g., an antimicrobial, anti-biofilm, antibacterial, anti-inflammatory, antifungal, antiviral, anti-autoimmune, anti-cancer, pro-apoptosis, anti-proliferative, and/or MMP modulating atomically disordered, nanocrystalline silver-containing material) can be contacted with the subject's ear to reduce the occurrence of ear-related conditions (e.g., swimmer's ear, inner ear infections, outer ear infections, middle ear infections) in the ear of the patient. The metal-containing material may be in the form of, for example, a foam, a solution (e.g., an injected solution), a mist, a sponge and/or a tape. Without wishing to be bound by theory, it is believed that this method reduces the occurrence of ear-related conditions in the subject by reducing the presence in the subject's ear of one or more pathogens of the ear-related conditions (e.g., one or more prions, parasites, fungi, viruses, inflammatory agents, cancer cells, allergens and/or bacteria) that can result in the ear-related conditions.

As explained below, the metal-containing material can be used to treat various subjects and conditions. As also explained below, the metal-containing material can be in any of a variety of forms when delivered to a subject, and the metal-containing material can be delivered to a subject in a variety of ways. In certain embodiments, however, the metal-containing material is not in the form of a dressing, the prophylactically treated condition is not a bacterial condition, and/or the metal-containing material is not contacted with the subject's skin.

Subjects

The metal-containing material can be used to treat, for example a human or an animal (e.g., a dog, a cat, a horse, a bird, a reptile, an amphibian, a fish, a turtle, a guinea pig, a hamster, a rodent, a cow, a pig, a goat, a primate, a monkey, a chicken, a turkey, a buffalo, an ostrich, a sheep, a llama).

Conditions

The conditions that can be treated with the metal-containing material include, for example, bacterial conditions, microbial conditions, biofilm conditions, inflammatory conditions, fungal conditions, viral conditions, autoimmune conditions, idiopathic conditions, hyperproliferative conditions, noncancerous growths and/or cancerous conditions (e.g., tumorous conditions, hematologic malignancies). Such conditions can be associated with, for example, one or more prions, parasites, fungi, viruses, inflammatory agents, cancer cells, allergens and/or bacteria. The conditions can be, for example, slow healing conditions, non-healing conditions or impaired healing conditions.

In some embodiments the condition can be a skin condition or an integument condition (e.g., a bacterial skin condition, a microbial skin condition, a biofilm skin condition, an inflammatory skin condition, a hyperproliferative skin condition, a fungal skin condition, a viral skin condition, an autoimmune skin condition, an idiopathic skin condition, a hyperproliferative skin condition, a cancerous skin condition, a microbial integument condition, an inflammatory integument condition, a fungal integument condition, a viral integument condition, an autoimmune integument condition, an idiopathic integument condition, a hyperproliferative integument condition, a cancerous integument condition). Examples of skin conditions or integument conditions include a burn, eczema (e.g., atopic eczema, acrodermatitis continua, contact allergic dermatitis, contact irritant dermatitis, dyshidrotic eczema, pompholyx, lichen simplex chronicus, nummular eczema, seborrheic dermatitis, stasis eczema), erythroderma, an insect bite, mycosis fungoides, pyoderma gangrenosum, eythrema multiforme, rosacea, onychomycosis, acne (e.g., acne vulgaris, neonatal acne, infantile acne, pomade acne), psoriasis, Reiter's syndrome, pityriasis rubra pilaris, hyperpigmentation, vitiligo, scarring conditions (e.g., hypertropic scarring), keloid, lichen planus, age-related skin disorder (e.g., wrinkles, cellulite) and hyperproliferative skin disorders, such as, for example, hyperproliferative variants of the disorders of keratinization (e.g., actinic keratosis, senile keratosis). As an example, the metal-containing material can be used prophylactically to reduce (e.g., prevent) the occurrence of a particular burn (e.g., a second degree burn) becoming a more severe burn (e.g., a third degree burn).

In certain embodiments, the condition can be a respiratory condition (e.g., a bacterial respiratory condition, a biofilm respiratory condition, a microbial respiratory condition, an inflammatory respiratory condition, a fungal respiratory condition, a viral respiratory condition, an autoimmune respiratory condition, an idiopathic respiratory condition, a hyperproliferative respiratory condition, a cancerous respiratory condition). Examples of respiratory conditions include asthma, emphysema, bronchitis, pulmonary edema, acute respiratory distress syndrome, bronchopulmonary dysplasia, fibrotic conditions (e.g., pulmonary fibrosis), pulmonary atelectasis, tuberculosis, pneumonia, sinusitis, allergic rhinitis, pharyngitis, mucositis, stomatitis, chronic obstructive pulmonary disease, bronchiectasis, lupus pneumonitis and cystic fibrosis.

In some embodiments, the condition can be a musculo-skeletal condition (e.g., a bacterial musculo-skeletal condition, a biofilm musculo-skeletal condition, a microbial musculo-skeletal condition, an inflammatory musculo-skeletal condition, a fungal musculo-skeletal condition, a viral musculo-skeletal condition, an autoimmune musculo-skeletal condition, an idiopathic musculo-skeletal condition, a hyperproliferative musculo-skeletal condition, a cancerous musculo-skeletal condition). A musculo-skeletal condition can be, for example, a degenerative musculo-skeletal condition (e.g., arthritis) or a traumatic musculo-skeletal condition (e.g., a torn or damaged muscle). Examples of musculo-skeletal conditions include tendonitis, osteomyelitis, fibromyalgia, bursitis and arthritis.

In certain embodiments, the condition can be a circulatory condition (e.g., a bacterial circulatory condition, a biofilm circulatory condition, a microbial circulatory condition, an inflammatory circulatory condition, a fungal circulatory condition, a viral circulatory condition, an autoimmune circulatory condition, an idiopathic circulatory condition, a hyperproliferative circulatory condition, a cancerous circulatory condition). As referred to herein, circulatory conditions include lymphatic conditions. Examples of circulatory conditions include arteriosclerosis, lymphoma, septicemia, leukemia, ischemic vascular disease, lymphangitis and atherosclerosis. Areas of the circulatory system include, for example, the heart, the lymphatic system, blood, blood vessels (e.g., arteries, veins).

In some embodiments, the condition can be a mucosal or serosal condition (e.g., a bacterial mucosal or serosal condition, a biofilm mucosal or serosal condition, a microbial mucosal or serosal condition, an inflammatory mucosal or serosal condition, a fungal mucosal or serosal condition, a viral mucosal or serosal condition, an autoimmune mucosal or serosal condition, an idiopathic mucosal or serosal condition, a hyperproliferative mucosal or serosal condition, a cancerous mucosal or serosal condition). Examples of mucosal or serosal conditions include pericarditis, Bowen's disease, stomatitis, prostatitis, sinusitis, allergic rhinitis, digestive disorders, peptic ulcers, esophageal ulcers, gastric ulcers, duodenal ulcers, espohagitis, gastritis, enteritis, enterogastric intestinal hemorrhage, toxic epidermal necrolysis syndrome, Stevens Johnson syndrome, cystic fibrosis, bronchitis, pneumonia (e.g., nosocomial pneumonia, ventilator-associated pneumonia), pharyngitis, common cold, ear infections, sore throat, sexually transmitted diseases (e.g., syphilis, gonorrhea, herpes, genital warts, HIV, chlamydia), inflammatory bowel disease, colitis, hemorrhoids, thrush, dental conditions, oral conditions, conjunctivitis, and periodontal conditions.

In some embodiments, the metal-containing material can be used to treat hyperproliferation of cell growth (e.g., cancerous conditions, such as malignant tumors, or non-cancerous conditions, such as benign tumors), the metal-containing material can be used to induce apoptosis (programmed cell death), modulate matrix metalloproteinases (MMPs) and/or modulate cytokines by contacting affected tissue (e.g., a hyperplastic tissue, a tumor tissue or a cancerous lesion) with the metal-containing material. It has been observed that the metal-containing material (e.g., an antimicrobial, anti-biofilm, antibacterial, anti-inflammatory, antifungal, antiviral, anti-autoimmune, anti-cancer, pro-apoptosis, anti-proliferative, and/or MMP modulating atomically disordered, silver-containing material) can be effective in preventing production of a high number of MMPs and/or cytokines by certain cells without necessarily reducing MMP and/or cytokine production by the same cells to about zero. It is believed, however, that in certain embodiments, the metal-containing material can be used to inhibit MMP and/or cytokine production (e.g., bring MMP and/or cytokine production to normal levels, desired levels, and/or about zero) in certain cells.

MMPs refer to any protease of the family of MMPs which are involved in the degradation of connective tissues, such as collagen, elastins, fibronectin, laminin, and other components of the extracellular matrix, and associated with conditions in which excessive degradation of extracellular matrix occurs, such as tumor invasion and metastasis. Examples of MMPs include MMP-2 (secreted by fibroblasts and a wide variety of other cell types) and MMP-9 (released by mononuclear phagocytes, neutrophils, corneal epithelial cells, tumor cells, cytotrophoblasts and keratinocytes). Cytokine refers to a nonimmunoglobulin polypeptide secreted by monocytes and lymphocytes in response to interaction with a specific antigen, a nonspecific antigen, or a nonspecific soluble stimulus (e.g., endotoxin, other cytokines). Cytokines affect the magnitude of inflammatory or immune responses. Cytokines can be divided into several groups, which include interferons, tumor necrosis factor (TNF), interleukins (IL-1 to IL-8), transforming growth factors, and the hematopoietic colony-stimulating factors. An example of a cytokine is TNF-α. A fibroblast is an area connective tissue cell which is a flat-elongated cell with cytoplasmic processes at each end having a flat, oval vesicular nucleus. Fibroblasts which differentiate into chondroblasts, collagenoblasts, and osteoblasts form the fibrous tissues in the body, tendons, aponeuroses, supporting and binding tissues of all sorts. Hyperplastic tissue refers to tissue in which there is an abnormal multiplication or increase in the number of cells in a normal arrangement in normal tissue or an organ. A tumor refers to spontaneous growth of tissue in which multiplication of cells is abnormal, uncontrolled and progressive. A tumor generally serves no useful function and grows at the expense of the healthy organism. A cancerous lesion is a tumor of epithelial tissue, or malignant, new growth made up of epithelial cells tending to infiltrate surrounding tissues and to give rise to metastases. As used in reference to the skin, a cancerous lesion means a lesion which may be a result of a primary cancer, or a metastasis to the site from a local tumor or from a tumor in a distant site. It may take the form of a cavity, an open area on the surface of the skin, skin nodules, or a nodular growth extending from the surface of the skin.

Conditions characterized by undesirable MMP activity include ulcers, asthma, acute respiratory distress syndrome, skin disorders, skin aging, keratoconus, restenosis, osteo- and rheumatoid arthritis, degenerative joint disease, bone disease, wounds, cancer including cell proliferation, invasiveness, metastasis (carcinoma, fibrosarcoma, osteosarcoma), hypovolemic shock, periodontal disease, epidermolysis bullosa, scleritis, atherosclerosis, multiple sclerosis, inflammatory diseases of the central nervous system, vascular leakage syndrome, collagenase induced disease, adhesions of the peritoneum, strictures of the esophagus or bowel, ureteral or urethral strictures, and biliary strictures. Excessive TNF production has been reported in diseases which are characterized by excessive MMP activity, such as autoimmune disease, cancer, cachexia, HIV infection, and cardiovascular conditions.

As an example, a subject may be treated prophylactically to reduce (e.g., prevent) a cancerous or precancerous lesion by contacting the affected area, or potentially affected area, with the metal-containing material (e.g., in the form of a solution, a mist, a dressing, a bandage, a tape, etc.). As another example, after operating on an area having a cancerous tumor, the area may be contacted with the metal-containing material (e.g., in the form of a solution, a mist, a bandage, a tape, etc.).

Materials

The metal-containing material can be an ionic material or a non-ionic material. The metal-containing material can be, for example, an atom, a molecule, or a cluster.

In general, the metal-containing material is a metal or an alloy. Examples of metal elements that can be contained in metal-containing materials include Group I A metal elements, Group II A metal elements, Group III A metal elements, Group IV A metal elements, Group V A metal elements, Group VI A metal elements, Group VII A metal elements, Group VIII A metal elements, Group I B metal elements, Group II B metal elements, members of the lanthanide metal element series, and members of the actinide metal elements series. In certain embodiments, metal-containing materials contain silver, gold, platinum, palladium, iridium, zinc, copper, tin, antimony, and/or bismuth. In some embodiments, a metal-containing material can include one or more transition metal elements (e.g., scandium, titanium, vanadium, chromium, manganese, iron, cobalt, nickel, copper and/or zinc). As an example, a metal-containing material can contain silver and platinum.

In addition to one or more metal elements, a metal-containing material can contain oxygen, nitrogen, carbon, boron, sulfur, phosphorus, silicon, a halogen (e.g., fluorine, chlorine, bromine, iodine) and/or hydrogen. Examples of such metal-containing materials include metal oxides, metal nitrides, metal carbides, metal borides, metal sulfides, metal nitrates, metal hydroxides, metal carbonates, metal sulfadiazines, metal hydrides, metal acetates, metal lactates, metal citrates, metal myristates, metal sorbates, metal stearates, metal oleates, metal glutonates, metal adipates, metal phosphides, metal silicates, alkali metal thiosulphates (e.g., sodium metal thiosulphate, potassium metal thiosulphate) and metal halides (e.g., metal fluorides, metal chlorides, metal bromides, metal iodides) and metal hydrides. In certain embodiments, a metal-containing material contains at least about one atomic percent (e.g., at least about three atomic percent, at least about five atomic percent, at least about 10 atomic percent, at least about 20 atomic percent, at least about 30 atomic percent, at least about 40 atomic percent, at least about 50 atomic percent) and/or at most about 90 atomic percent (e.g., at most about 80 atomic percent, at most about 70 atomic percent, at most about 60 atomic percent, at most about 50 atomic percent, at most about 40 atomic percent, at most about 30 atomic percent, at most about 20 atomic percent, at most about 15 atomic percent, at most about 12 atomic percent, at most about 10 atomic percent) of nonmetallic elements. For example, in some embodiments, a silver-containing material can contain oxygen in an amount from about five atomic percent to about 20 atomic percent (e.g., from about five atomic percent to about 15 atomic percent, from about eight atomic percent to about 12 atomic percent).

In some embodiments, the metal-containing material can be a noble metal (e.g., silver nitrate, silver hydroxide, silver sulfadiazine, colloidal silver, silver carbonate, silver oxide, silver acetate, silver lactate, silver citrate, silver succinate, silver chlorate, silver sorbate, silver myristate, silver stearate, silver oleate, silver glutonate, silver adipate, alkali silver thiosulphate (e.g., sodium silver thiosulphate, potassium silver thiosulphate).

In some embodiments, a metal-containing material can have a prophylactic ratio of about 0.95 or less (e.g., about 0.9 or less, about 0.8 or less, about 0.7 or less, about 0.6 or less, about 0.5 or less, about 0.4 or less, about 0.3 or less, about 0.2 or less, about 0.1 or less, about 0.05 or less). The prophylactic ratio of a material refers to the ratio of the probability of a subject contracting a condition when treated with the material to the probability of the subject contracting the condition without being treated with the material.

In certain embodiments, the metal-containing materials is an antimicrobial material, an anti-biofilm material, an antibacterial material, an anti-inflammatory material, an antifungal material, an antiviral material, an anti-autoimmune material, an anti-cancer material, a pro-apoptosis material, anti-proliferative, an MMP modulating material, an atomically disordered crystalline material, and/or a nanocrystalline material.

As used herein, an antimicrobial material herein refers to a material that has sufficient antimicrobial activity to have a beneficial therapeutic effect. In certain embodiments, an antimicrobial material has a corrected zone of inhibition (“CZOI”) of at least about two millimeters (e.g., at least about three millimeters, at least about four millimeters, at least about five millimeters, at least about six millimeters, at least about seven millimeters, at least about eight millimeters, at least about nine millimeters, at least about 10 millimeters). The CZOI of a material is determined as follows. The material is formed as a coating on a dressing (see discussion below). Basal medium Eagle (BME) with Earle's salts and L-glutamine is modified with calf/serum (10%) and 1.5% agar prior to being dispensed (15 ml) into Petri dishes. The agar containing Petri dishes are allowed to surface dry prior to being inoculated with a lawn of Staphylococcus aureus ATCC #25923. The inoculant is prepared from Bactrol Discs (Difco, M.) which are reconstituted as per the manufacturer's directions. Immediately after inoculation, the coatings to be tested are placed on the surface of the agar. The dishes are incubated for 24 hours at 37° C. After this incubation period, the zone of inhibition (“ZOI”) is measured and the CZOI is calculated as the ZOI minus the diameter of the test material in contact with the agar. It is to be noted that, while this test for antimicrobial properties is performed on materials that are in the form of a coating on a substrate (e.g., in the form of a dressing), antimicrobial materials are not limited to materials that are coated on a substrate. Rather, a material in any form may be antimicrobial, but it is in the form of a coating on a substrate (e.g., in the form of a dressing) when its antimicrobial properties are tested according to the procedure described herein.

As referred to herein, an atomically disordered, crystalline material (e.g., an atomically disordered, nanocrystalline material) means a material that has more long range ordered, crystalline structure (a lesser degree of defects) than the material has in a fully amorphous state, but that also has less long range, ordered crystalline structure (a higher degree of defects) than the material has in a bulk crystalline state, such as in the form of a cast, wrought or plated material. Examples of defects include point defects, vacancies, line defects, grain boundaries, subgrain boundaries and amorphous regions. Point defects are defects on a size scale of no more than about four atomic spacings. A vacancy is the omission of an atom from its regular atomic site in the crystal lattice. Line defects are defective regions (e.g., edge dislocations, screw dislocations) that result in lattice distortions along a line (which may or may not be a straight line), and generally have a longer scale than point defects. In an edge dislocation, a lattice displacement is produced by a plane of atoms that forms a terminus of the lattice. In a screw dislocation, part of the lattice is displaced with respect to an adjacent part of the lattice. Grain boundaries separate regions having different crystallographic orientation or misorientation (e.g., high angle grain boundaries, low angle grain boundaries, including tilt boundaries and twist boundaries). Subgrain boundaries refer to low angle grain boundaries. An amorphous region is a region that does not exhibit long range, ordered crystalline structure. In certain embodiments, an atomically disordered, crystalline material (e.g., an atomically disordered, nanocrystalline material) has a degree of atomic disorder that is about the same as the degree of atomic disorder of the nanocrystalline silver coating of a member of the Acticoat® family of dressings (Smith & Nephew, Hull, UK) (e.g., an Acticoat® dressing, an Acticoat® dressing, an Acticoat® moisture coating dressing, an Acticoat® absorbent dressings). In some embodiments, an atomically disordered, crystalline material (e.g., an atomically disordered, nanocrystalline material) has a degree of atomic disorder that is about the same as the degree of atomic disorder of the nanocrystalline silver coatings having a CZOI of at least five millimeters that are disclosed in the examples of Burrell et al., U.S. Pat. No. 5,958,440. In certain embodiments, an atomically disordered, crystalline material (e.g., an atomically disordered, nanocrystalline material), when contacted with an alcohol or water-based electrolyte, is released into the alcohol or water-based electrolyte (e.g., as ions, atoms, molecules and/or clusters) over a time scale of at least about one hour (e.g., at least about two hours, at least about 10 hours, at least about a day). Examples of alcohols and/or water-based electrolytes include body fluids (e.g., blood, urine, saliva) and body tissue (e.g., skin, muscle, bone).

As referred to herein, a nanocrystalline material is a single-phase polycrystal or a multi-phase polycrystal having a maximum dimension of about 100 nanometers or less (e.g., about 90 nanometers or less, about 80 nanometers or less, about 70 nanometers or less, about 60 nanometers or less, about 50 nanometers or less, about 40 nanometers or less, about 30 nanometers or less, about 25 nanometers or less) in at least one dimension.

Examples of antimicrobial metal-containing materials (which may or may not also be an atomically disordered crystalline material or a nanocrystalline material) include antimicrobial silver-containing materials (e.g., antimicrobial silver, antimicrobial silver alloys, antimicrobial silver oxides, antimicrobial silver carbides, antimicrobial silver nitrides, antimicrobial silver borides, antimicrobial silver sulfides, antimicrobial silver myristates, antimicrobial silver stearates, antimicrobial silver oleates, antimicrobial silver glutonates, antimicrobial silver glutonates, antimicrobial silver adipates, antimicrobial silver silicates, antimicrobial silver phosphides, antimicrobial silver halides, antimicrobial silver hydrides, antimicrobial silver nitrates, antimicrobial silver hydroxides, antimicrobial silver carbonates, antimicrobial silver sulfadiazines, antimicrobial silver acetates, antimicrobial silver lactates, antimicrobial silver citrates, antimicrobial alkali silver thiosulphates (e.g., antimicrobial sodium silver thiosulphate, antimicrobial potassium silver thiosulphate)), antimicrobial gold-containing materials (e.g., antimicrobial gold, antimicrobial gold alloys, antimicrobial gold oxides, antimicrobial gold carbides, antimicrobial gold nitrides, antimicrobial gold borides, antimicrobial gold sulfides, antimicrobial gold myristates, antimicrobial gold stearates, antimicrobial gold oleates, antimicrobial gold glutonates, antimicrobial gold glutonates, antimicrobial gold adipates, antimicrobial gold silicates, antimicrobial gold phosphides, antimicrobial gold halides, antimicrobial gold hydrides, antimicrobial gold nitrates, antimicrobial gold hydroxides, antimicrobial gold carbonates, antimicrobial gold sulfadiazines, antimicrobial gold acetates, antimicrobial gold lactates, antimicrobial gold citrates, antimicrobial alkali gold thiosulphates (e.g., antimicrobial sodium gold thiosulphate, antimicrobial potassium gold thiosulphate)), antimicrobial platinum-containing materials (e.g., antimicrobial platinum, antimicrobial platinum alloys, antimicrobial platinum oxides, antimicrobial platinum carbides, antimicrobial platinum nitrides, antimicrobial platinum borides, antimicrobial platinum sulfides, antimicrobial platinum myristates, antimicrobial platinum stearates, antimicrobial platinum oleates, antimicrobial platinum glutonates, antimicrobial platinum glutonates, antimicrobial platinum adipates, antimicrobial platinum silicates, antimicrobial platinum phosphides, antimicrobial platinum halides, antimicrobial platinum hydrides, antimicrobial platinum nitrates, antimicrobial platinum hydroxides, antimicrobial platinum carbonates, antimicrobial platinum sulfadiazines, antimicrobial platinum acetates, antimicrobial platinum lactates, antimicrobial platinum citrates, antimicrobial alkali platinum thiosulphates (e.g., antimicrobial sodium platinum thiosulphate, antimicrobial potassium platinum thiosulphate)), antimicrobial palladium-containing materials (e.g., antimicrobial palladium, antimicrobial palladium alloys, antimicrobial palladium oxides, antimicrobial palladium carbides, antimicrobial palladium nitrides, antimicrobial palladium borides, antimicrobial palladium sulfides, antimicrobial palladium myristates, antimicrobial palladium stearates, antimicrobial palladium oleates, antimicrobial palladium glutonates, antimicrobial palladium glutonates, antimicrobial palladium adipates, antimicrobial palladium silicates, antimicrobial palladium phosphides, antimicrobial palladium halides, antimicrobial palladium hydrides, antimicrobial palladium nitrates, antimicrobial palladium hydroxides, antimicrobial palladium carbonates, antimicrobial palladium sulfadiazines, antimicrobial palladium acetates, antimicrobial palladium lactates, antimicrobial palladium citrates, antimicrobial alkali palladium thiosulphates (e.g., antimicrobial sodium palladium thiosulphate, antimicrobial potassium palladium thiosulphate)), antimicrobial iridium-containing materials (e.g., antimicrobial iridium, antimicrobial iridium alloys, antimicrobial iridium oxides, antimicrobial iridium carbides, antimicrobial iridium nitrides, antimicrobial iridium borides, antimicrobial iridium sulfides, antimicrobial iridium myristates, antimicrobial iridium stearates, antimicrobial iridium oleates, antimicrobial iridium glutonates, antimicrobial iridium glutonates, antimicrobial iridium adipates, antimicrobial iridium silicates, antimicrobial iridium phosphides, antimicrobial iridium halides, antimicrobial iridium hydrides, antimicrobial iridium nitrates, antimicrobial iridium hydroxides, antimicrobial iridium carbonates, antimicrobial iridium sulfides, antimicrobial iridium sulfadiazines, antimicrobial iridium acetates, antimicrobial iridium lactates, antimicrobial iridium citrates, antimicrobial alkali iridium thiosulphates (e.g., antimicrobial sodium iridium thiosulphate, antimicrobial potassium iridium thiosulphate)), antimicrobial zinc-containing materials (e.g., antimicrobial zinc, antimicrobial zinc alloys, antimicrobial zinc oxides, antimicrobial zinc carbides, antimicrobial zinc nitrides, antimicrobial zinc borides, antimicrobial zinc sulfides, antimicrobial zinc myristates, antimicrobial zinc stearates, antimicrobial zinc oleates, antimicrobial zinc glutonates, antimicrobial zinc glutonates, antimicrobial zinc adipates, antimicrobial zinc silicates, antimicrobial zinc phosphides, antimicrobial zinc halides, antimicrobial zinc hydrides, antimicrobial zinc nitrates, antimicrobial zinc hydroxides, antimicrobial zinc carbonates, antimicrobial zinc sulfides, antimicrobial zinc sulfadiazines, antimicrobial zinc acetates, antimicrobial zinc lactates, antimicrobial zinc citrates, antimicrobial alkali zinc thiosulphates (e.g., antimicrobial sodium zinc thiosulphate, antimicrobial potassium zinc thiosulphate)), antimicrobial copper-containing materials (e.g., antimicrobial copper, antimicrobial copper alloys, antimicrobial copper oxides, antimicrobial copper carbides, antimicrobial copper nitrides, antimicrobial copper borides, antimicrobial copper sulfides, antimicrobial copper myristates, antimicrobial copper stearates, antimicrobial copper oleates, antimicrobial copper glutonates, antimicrobial copper glutonates, antimicrobial copper adipates, antimicrobial copper silicates, antimicrobial copper phosphides, antimicrobial copper halides, antimicrobial copper hydrides, antimicrobial copper nitrates, antimicrobial copper hydroxides, antimicrobial copper carbonates, antimicrobial copper sulfides, antimicrobial copper sulfadiazines, antimicrobial copper acetates, antimicrobial copper lactates, antimicrobial copper citrates, antimicrobial alkali copper thiosulphates (e.g., antimicrobial sodium copper thiosulphate, antimicrobial potassium copper thiosulphate)), antimicrobial tin-containing materials (e.g., antimicrobial tin, antimicrobial tin alloys, antimicrobial tin oxides, antimicrobial tin carbides, antimicrobial tin nitrides, antimicrobial tin borides, antimicrobial tin sulfides, antimicrobial tin myristates, antimicrobial tin stearates, antimicrobial tin oleates, antimicrobial tin glutonates, antimicrobial tin glutonates, antimicrobial tin adipates, antimicrobial tin silicates, antimicrobial tin phosphides, antimicrobial tin halides, antimicrobial tin hydrides, antimicrobial tin nitrates, antimicrobial tin hydroxides, antimicrobial tin carbonates, antimicrobial tin sulfides, antimicrobial tin sulfadiazines, antimicrobial tin acetates, antimicrobial tin lactates, antimicrobial tin citrates, antimicrobial alkali tin thiosulphates (e.g., antimicrobial sodium tin thiosulphate, antimicrobial potassium tin thiosulphate)), antimicrobial antimony-containing materials (e.g., antimicrobial antimony, antimicrobial antimony alloys, antimicrobial antimony oxides, antimicrobial antimony carbides, antimicrobial antimony nitrides, antimicrobial antimony borides, antimicrobial antimony sulfides, antimicrobial antimony myristates, antimicrobial antimony stearates, antimicrobial antimony oleates, antimicrobial antimony glutonates, antimicrobial antimony glutonates, antimicrobial antimony adipates, antimicrobial antimony silicates, antimicrobial antimony phosphides, antimicrobial antimony halides, antimicrobial antimony hydrides, antimicrobial antimony nitrates, antimicrobial antimony hydroxides, antimicrobial antimony carbonates, antimicrobial antimony sulfides, antimicrobial antimony sulfadiazines, antimicrobial antimony acetates, antimicrobial antimony lactates, antimicrobial antimony citrates, antimicrobial alkali antimony thiosulphates (e.g., antimicrobial sodium antimony thiosulphate, antimicrobial potassium antimony thiosulphate)), antimicrobial bismuth containing materials (e.g., antimicrobial bismuth, antimicrobial bismuth alloys, antimicrobial bismuth oxides, antimicrobial bismuth carbides, antimicrobial bismuth nitrides, antimicrobial bismuth borides, antimicrobial bismuth sulfides, antimicrobial bismuth myristates, antimicrobial bismuth stearates, antimicrobial bismuth oleates, antimicrobial bismuth glutonates, antimicrobial bismuth glutonates, antimicrobial bismuth adipates, antimicrobial bismuth silicates, antimicrobial bismuth phosphides, antimicrobial bismuth halides, antimicrobial bismuth hydrides, antimicrobial bismuth nitrates, antimicrobial bismuth hydroxides, antimicrobial bismuth carbonates, antimicrobial bismuth sulfides, antimicrobial bismuth sulfadiazines, antimicrobial bismuth acetates, antimicrobial bismuth lactates, antimicrobial bismuth citrates, antimicrobial alkali bismuth thiosulphates (e.g., antimicrobial sodium bismuth thiosulphate, antimicrobial potassium bismuth thiosulphate)).

While the preceding paragraph lists certain metal-containing materials that are anti-microbial, similar metal-containing compounds (oxides, carbides, nitrides, borides, sulfides, myristates, stearates, oleates, glutonates, adipates, silicates, phosphides, halides, hydrides, nitrates, hydroxides, carbonates, sulfides, sulfadiazines, acetates, lactates, citrates and/or alkali metal thiosulphates of silver, gold, palladium, platinum, tin, iridium, antimony, bismuth, copper, zinc) can be anti-biofilm materials, antibacterial materials, anti-inflammatory materials, antifungal materials, antiviral materials, anti-autoimmune materials, anti-cancer materials, pro-apoptosis materials, anti-proliferatives, and/or MMP modulating materials.

Examples of nanocrystalline metal-containing materials (which may or may not also be an antimicrobial material or an atomically disordered crystalline material) include nanocrystalline silver-containing materials (e.g., nanocrystalline silver, nanocrystalline silver alloys, nanocrystalline silver oxides, nanocrystalline silver hydroxides, nanocrystalline silver carbides, nanocrystalline silver nitrides, nanocrystalline silver borides, nanocrystalline silver sulfides, nanocrystalline silver halides, nanocrystalline silver myristates, nanocrystalline silver stearates, nanocrystalline silver oleates, nanocrystalline silver glutonates, nanocrystalline silver glutonates, nanocrystalline silver adipates, nanocrystalline silver silicates, nanocrystalline silver phosphides, nanocrystalline silver hydrides, nanocrystalline silver nitrates, nanocrystalline silver carbonates, nanocrystalline silver sulfides, nanocrystalline silver sulfadiazines, nanocrystalline silver acetates, nanocrystalline silver lactates, nanocrystalline silver citrates, nanocrystalline alkali silver thiosulphates (e.g., nanocrystalline sodium silver thiosulphate, nanocrystalline potassium silver thiosulphate)), nanocrystalline gold-containing materials (e.g., nanocrystalline gold, nanocrystalline gold alloys, nanocrystalline gold oxides, nanocrystalline gold hydroxides, nanocrystalline gold carbides, nanocrystalline gold nitrides, nanocrystalline gold borides, nanocrystalline gold sulfides, nanocrystalline gold halides, nanocrystalline gold hydrides, nanocrystalline gold nitrates, nanocrystalline gold myristates, nanocrystalline gold stearates, nanocrystalline gold oleates, nanocrystalline gold glutonates, nanocrystalline gold glutonates, nanocrystalline gold adipates, nanocrystalline gold silicates, nanocrystalline gold phosphides, nanocrystalline gold carbonates, nanocrystalline gold sulfides, nanocrystalline gold sulfadiazines, nanocrystalline gold acetates, nanocrystalline gold lactates, nanocrystalline gold citrates, nanocrystalline alkali gold thiosulphates (e.g., nanocrystalline sodium gold thiosulphate, nanocrystalline potassium gold thiosulphate)), nanocrystalline platinum-containing materials (e.g., nanocrystalline platinum, nanocrystalline platinum alloys, nanocrystalline platinum oxides, nanocrystalline platinum hydroxides, nanocrystalline platinum carbides, nanocrystalline platinum nitrides, nanocrystalline platinum borides, nanocrystalline platinum sulfides, nanocrystalline platinum myristates, nanocrystalline platinum stearates, nanocrystalline platinum oleates, nanocrystalline platinum glutonates, nanocrystalline platinum glutonates, nanocrystalline platinum adipates, nanocrystalline platinum silicates, nanocrystalline platinum phosphides, nanocrystalline platinum halides, nanocrystalline platinum hydrides, nanocrystalline platinum nitrates, nanocrystalline platinum carbonates, nanocrystalline platinum sulfides, nanocrystalline platinum sulfadiazines, nanocrystalline platinum acetates, nanocrystalline platinum lactates, nanocrystalline platinum citrates, nanocrystalline alkali platinum thiosulphates (e.g., nanocrystalline sodium platinum thiosulphate, nanocrystalline potassium platinum thiosulphate)), nanocrystalline palladium-containing materials (e.g., nanocrystalline palladium, nanocrystalline palladium alloys, nanocrystalline palladium oxides, nanocrystalline palladium hydroxides, nanocrystalline palladium carbides, nanocrystalline palladium nitrides, nanocrystalline palladium borides, nanocrystalline palladium sulfides, nanocrystalline palladium myristates, nanocrystalline palladium stearates, nanocrystalline palladium oleates, nanocrystalline palladium glutonates, nanocrystalline palladium glutonates, nanocrystalline palladium adipates, nanocrystalline palladium silicates, nanocrystalline palladium phosphides, nanocrystalline palladium halides, nanocrystalline palladium hydrides, nanocrystalline palladium nitrates, nanocrystalline palladium carbonates, nanocrystalline palladium sulfides, nanocrystalline palladium sulfadiazines, nanocrystalline palladium acetates, nanocrystalline palladium lactates, nanocrystalline palladium citrates, nanocrystalline alkali palladium thiosulphates (e.g., nanocrystalline sodium palladium thiosulphate, nanocrystalline potassium palladium thiosulphate)), nanocrystalline iridium-containing materials (e.g., nanocrystalline iridium, nanocrystalline iridium alloys, nanocrystalline iridium oxides, nanocrystalline iridium hydroxides, nanocrystalline iridium carbides, nanocrystalline iridium nitrides, nanocrystalline iridium borides, nanocrystalline iridium sulfides, nanocrystalline iridium myristates, nanocrystalline iridium stearates, nanocrystalline iridium oleates, nanocrystalline iridium glutonates, nanocrystalline iridium glutonates, nanocrystalline iridium adipates, nanocrystalline iridium silicates, nanocrystalline iridium phosphides, nanocrystalline iridium halides, nanocrystalline iridium hydrides, nanocrystalline iridium nitrates, nanocrystalline iridium carbonates, nanocrystalline iridium sulfides, nanocrystalline iridium sulfadiazines, nanocrystalline iridium acetates, nanocrystalline iridium lactates, nanocrystalline iridium citrates, nanocrystalline alkali iridium thiosulphates (e.g., nanocrystalline sodium iridium thiosulphate, nanocrystalline potassium iridium thiosulphate)), nanocrystalline zinc-containing materials (e.g., nanocrystalline zinc, nanocrystalline zinc alloys, nanocrystalline zinc oxides, nanocrystalline zinc hydroxides, nanocrystalline zinc carbides, nanocrystalline zinc nitrides, nanocrystalline zinc borides, nanocrystalline zinc sulfides, nanocrystalline zinc myristates, nanocrystalline zinc stearates, nanocrystalline zinc oleates, nanocrystalline zinc glutonates, nanocrystalline zinc glutonates, nanocrystalline zinc adipates, nanocrystalline zinc silicates, nanocrystalline zinc phosphides, nanocrystalline zinc halides, nanocrystalline zinc hydrides, nanocrystalline zinc nitrates, nanocrystalline zinc carbonates, nanocrystalline zinc sulfides, nanocrystalline zinc sulfadiazines, nanocrystalline zinc acetates, nanocrystalline zinc lactates, nanocrystalline zinc citrates, nanocrystalline alkali zinc thiosulphates (e.g., nanocrystalline sodium zinc thiosulphate, nanocrystalline potassium zinc thiosulphate)), nanocrystalline copper-containing materials (e.g., nanocrystalline copper, nanocrystalline copper alloys, nanocrystalline copper oxides, nanocrystalline copper hydroxides, nanocrystalline copper carbides, nanocrystalline copper nitrides, nanocrystalline copper borides, nanocrystalline copper sulfides, nanocrystalline copper myristates, nanocrystalline copper stearates, nanocrystalline copper oleates, nanocrystalline copper glutonates, nanocrystalline copper glutonates, nanocrystalline copper adipates, nanocrystalline copper silicates, nanocrystalline copper phosphides, nanocrystalline copper halides, nanocrystalline copper hydrides, nanocrystalline copper nitrates, nanocrystalline copper carbonates, nanocrystalline copper sulfadiazines, nanocrystalline copper acetates, nanocrystalline copper lactates, nanocrystalline copper citrates, nanocrystalline alkali copper thiosulphates (e.g., nanocrystalline sodium copper thiosulphate, nanocrystalline potassium copper thiosulphate)), nanocrystalline tin-containing materials (e.g., nanocrystalline tin, nanocrystalline tin alloys, nanocrystalline tin oxides, nanocrystalline tin hydroxides, nanocrystalline tin carbides, nanocrystalline tin nitrides, nanocrystalline tin borides, nanocrystalline tin sulfides, nanocrystalline tin myristates, nanocrystalline tin stearates, nanocrystalline tin oleates, nanocrystalline tin glutonates, nanocrystalline tin glutonates, nanocrystalline tin adipates, nanocrystalline tin silicates, nanocrystalline tin phosphides, nanocrystalline tin halides, nanocrystalline tin hydrides, nanocrystalline tin nitrates, nanocrystalline tin carbonates, nanocrystalline tin sulfides, nanocrystalline tin sulfadiazines, nanocrystalline tin acetates, nanocrystalline tin lactates, nanocrystalline tin citrates, nanocrystalline alkali tin thiosulphates (e.g., nanocrystalline sodium tin thiosulphate, nanocrystalline potassium tin thiosulphate)), nanocrystalline antimony-containing materials (e.g., nanocrystalline antimony, nanocrystalline antimony alloys, nanocrystalline antimony oxides, nanocrystalline antimony hydroxides, nanocrystalline antimony carbides, nanocrystalline antimony nitrides, nanocrystalline antimony borides, nanocrystalline antimony sulfides, nanocrystalline antimony myristates, nanocrystalline antimony stearates, nanocrystalline antimony oleates, nanocrystalline antimony glutonates, nanocrystalline antimony glutonates, nanocrystalline antimony adipates, nanocrystalline antimony silicates, nanocrystalline antimony phosphides, nanocrystalline antimony halides, nanocrystalline antimony hydrides, nanocrystalline antimony nitrates, nanocrystalline antimony carbonates, nanocrystalline antimony sulfides, nanocrystalline antimony sulfadiazines, nanocrystalline antimony acetates, nanocrystalline antimony lactates, nanocrystalline antimony citrates, nanocrystalline alkali antimony thiosulphates (e.g., nanocrystalline sodium antimony thiosulphate, nanocrystalline potassium antimony thiosulphate)), nanocrystalline bismuth containing materials (e.g., nanocrystalline bismuth, nanocrystalline bismuth alloys, nanocrystalline bismuth oxides, nanocrystalline bismuth hydroxides, nanocrystalline bismuth carbides, nanocrystalline bismuth nitrides, nanocrystalline bismuth borides, nanocrystalline bismuth sulfides, nanocrystalline bismuth myristates, nanocrystalline bismuth stearates, nanocrystalline bismuth oleates, nanocrystalline bismuth glutonates, nanocrystalline bismuth glutonates, nanocrystalline bismuth adipates, nanocrystalline bismuth silicates, nanocrystalline bismuth phosphides, nanocrystalline bismuth halides, nanocrystalline bismuth hydrides, nanocrystalline bismuth nitrates, nanocrystalline bismuth carbonates, nanocrystalline bismuth sulfides, nanocrystalline anti bismuth sulfadiazines, nanocrystalline bismuth acetates, nanocrystalline bismuth lactates, nanocrystalline bismuth citrates, nanocrystalline alkali bismuth thiosulphates (e.g., nanocrystalline sodium bismuth thiosulphate, nanocrystalline potassium bismuth thiosulphate)).

Examples of atomically disordered, crystalline metal-containing material (which may or may not also be an antimicrobial material or a nanocrystalline material) include atomically disordered, crystalline silver-containing materials (e.g., atomically disordered, crystalline silver; atomically disordered, crystalline silver alloys; atomically disordered, crystalline silver oxides; atomically disordered, crystalline silver hydroxides; atomically disordered, crystalline silver carbides; atomically disordered, crystalline silver nitrides; atomically disordered, crystalline silver borides; atomically disordered, crystalline silver sulfides; atomically disordered, crystalline silver myristates; atomically disordered, crystalline silver stearates; atomically disordered, crystalline silver oleates; atomically disordered, crystalline silver glutonates; atomically disordered, crystalline silver glutonates; atomically disordered, crystalline silver adipates; atomically disordered, crystalline silver silicates; atomically disordered, crystalline silver phosphides; atomically disordered, crystalline silver halides; atomically disordered, crystalline silver hydrides, atomically disordered, crystalline silver nitrates; atomically disordered, crystalline silver carbonates; atomically disordered, crystalline silver sulfides; atomically disordered, crystalline silver sulfadiazines; atomically disordered, crystalline silver acetates; atomically disordered, crystalline silver lactates; atomically disordered, crystalline silver citrates; atomically disordered, crystalline alkali silver thiosulphates (e.g., atomically disordered, crystalline sodium silver thiosulphate, atomically disordered, crystalline potassium silver thiosulphate)), atomically disordered, crystalline gold-containing materials (atomically disordered, crystalline gold; atomically disordered, crystalline gold alloys; atomically disordered, crystalline gold oxides; atomically disordered, crystalline gold hydroxides; atomically disordered, crystalline gold carbides; atomically disordered, crystalline gold nitrides; atomically disordered, crystalline gold borides; atomically disordered, crystalline gold sulfides; atomically disordered, crystalline gold myristates; atomically disordered, crystalline gold stearates; atomically disordered, crystalline gold oleates; atomically disordered, crystalline gold glutonates; atomically disordered, crystalline gold glutonates; atomically disordered, crystalline gold adipates; atomically disordered, crystalline gold silicates; atomically disordered, crystalline gold phosphides; atomically disordered, crystalline gold halides; atomically disordered, crystalline gold hydrides, atomically disordered, crystalline gold nitrates; atomically disordered, crystalline gold carbonates; atomically disordered, crystalline gold sulfides; atomically disordered, crystalline gold sulfadiazines; atomically disordered, crystalline gold acetates; atomically disordered, crystalline gold lactates; atomically disordered, crystalline gold citrates; atomically disordered, crystalline alkali gold thiosulphates (e.g., atomically disordered, crystalline sodium gold thiosulphate, atomically disordered, crystalline potassium gold thiosulphate)), atomically disordered, crystalline platinum-containing materials (e.g., atomically disordered, crystalline platinum; atomically disordered, crystalline platinum alloys; atomically disordered, crystalline platinum oxides; atomically disordered, crystalline platinum hydroxides; atomically disordered, crystalline platinum carbides; atomically disordered, crystalline platinum nitrides; atomically disordered, crystalline platinum borides; atomically disordered, crystalline platinum sulfides; atomically disordered, crystalline platinum myristates; atomically disordered, crystalline platinum stearates; atomically disordered, crystalline platinum oleates; atomically disordered, crystalline platinum glutonates; atomically disordered, crystalline platinum glutonates; atomically disordered, crystalline platinum adipates; atomically disordered, crystalline platinum silicates; atomically disordered, crystalline platinum phosphides; atomically disordered, crystalline platinum halides; atomically disordered, crystalline platinum hydrides, atomically disordered, crystalline platinum nitrates; atomically disordered, crystalline platinum carbonates; atomically disordered, crystalline platinum sulfides; atomically disordered, crystalline platinum sulfadiazines; atomically disordered, crystalline platinum acetates; atomically disordered, crystalline platinum lactates; atomically disordered, crystalline platinum citrates; atomically disordered, crystalline alkali platinum thiosulphates (e.g., atomically disordered, crystalline sodium platinum thiosulphate, atomically disordered, crystalline potassium platinum thiosulphate), atomically disordered, crystalline palladium-containing materials (e.g., atomically disordered, crystalline palladium; atomically disordered, crystalline palladium alloys; atomically disordered, crystalline palladium oxides; atomically disordered, crystalline palladium hydroxides; atomically disordered, crystalline palladium carbides; atomically disordered, crystalline palladium nitrides; atomically disordered, crystalline palladium borides; atomically disordered, crystalline palladium sulfides; atomically disordered, crystalline palladium myristates; atomically disordered, crystalline palladium stearates; atomically disordered, crystalline palladium oleates; atomically disordered, crystalline palladium glutonates; atomically disordered, crystalline palladium glutonates; atomically disordered, crystalline palladium adipates; atomically disordered, crystalline palladium silicates; atomically disordered, crystalline palladium phosphides; atomically disordered, crystalline palladium halides; atomically disordered, crystalline palladium hydrides, atomically disordered, crystalline palladium nitrates; atomically disordered, crystalline palladium carbonates; atomically disordered, crystalline palladium sulfides; atomically disordered, crystalline palladium sulfadiazines; atomically disordered, crystalline palladium acetates; atomically disordered, crystalline palladium lactates; atomically disordered, crystalline palladium citrates; atomically disordered, crystalline alkali palladium thiosulphates (e.g., atomically disordered, crystalline sodium palladium thiosulphate, atomically disordered, crystalline potassium palladium thiosulphate)), atomically disordered, crystalline iridium-containing materials (e.g., atomically disordered, crystalline iridium; atomically disordered, crystalline iridium alloys; atomically disordered, crystalline iridium oxides; atomically disordered, crystalline iridium hydroxides; atomically disordered, crystalline iridium carbides; atomically disordered, crystalline iridium nitrides; atomically disordered, crystalline iridium borides; atomically disordered, crystalline iridium sulfides; atomically disordered, crystalline iridium myristates; atomically disordered, crystalline iridium stearates; atomically disordered, crystalline iridium oleates; atomically disordered, crystalline iridium glutonates; atomically disordered, crystalline iridium glutonates; atomically disordered, crystalline iridium adipates; atomically disordered, crystalline iridium silicates; atomically disordered, crystalline iridium phosphides; atomically disordered, crystalline iridium halides; atomically disordered, crystalline iridium hydrides, atomically disordered, crystalline iridium nitrates; atomically disordered, crystalline iridium carbonates; atomically disordered, crystalline iridium sulfides; atomically disordered, crystalline iridium sulfadiazines; atomically disordered, crystalline iridium acetates; atomically disordered, crystalline iridium lactates; atomically disordered, crystalline iridium citrates; atomically disordered, crystalline alkali iridium thiosulphates (e.g., atomically disordered, crystalline sodium iridium thiosulphate, atomically disordered, crystalline potassium iridium thiosulphate)), atomically disordered, crystalline zinc-containing materials (e.g., atomically disordered, crystalline zinc; atomically disordered, crystalline zinc alloys; atomically disordered, crystalline zinc oxides; atomically disordered, crystalline zinc hydroxides; atomically disordered, crystalline zinc carbides; atomically disordered, crystalline zinc nitrides; atomically disordered, crystalline zinc borides; atomically disordered, crystalline zinc sulfides; atomically disordered, crystalline zinc myristates; atomically disordered, crystalline zinc stearates; atomically disordered, crystalline zinc oleates; atomically disordered, crystalline zinc glutonates; atomically disordered, crystalline zinc glutonates; atomically disordered, crystalline zinc adipates; atomically disordered, crystalline zinc silicates; atomically disordered, crystalline zinc phosphides; atomically disordered, crystalline zinc halides; atomically disordered, crystalline zinc hydrides, atomically disordered, crystalline zinc nitrates; atomically disordered, crystalline zinc carbonates; atomically disordered, crystalline zinc sulfides; atomically disordered, crystalline zinc sulfadiazines; atomically disordered, crystalline zinc acetates; atomically disordered, crystalline zinc lactates; atomically disordered, crystalline zinc citrates; atomically disordered, crystalline alkali zinc thiosulphates (e.g., atomically disordered, crystalline sodium zinc thiosulphate, atomically disordered, crystalline potassium zinc thiosulphate)), atomically disordered, crystalline copper-containing materials (e.g., atomically disordered, crystalline copper; atomically disordered, crystalline copper alloys; atomically disordered, crystalline copper oxides; atomically disordered, crystalline copper hydroxides; atomically disordered, crystalline copper carbides; atomically disordered, crystalline copper nitrides; atomically disordered, crystalline copper borides; atomically disordered, crystalline copper sulfides; atomically disordered, crystalline copper myristates; atomically disordered, crystalline copper stearates; atomically disordered, crystalline copper oleates; atomically disordered, crystalline copper glutonates; atomically disordered, crystalline copper glutonates; atomically disordered, crystalline copper adipates; atomically disordered, crystalline copper silicates; atomically disordered, crystalline copper phosphides; atomically disordered, crystalline copper halides; atomically disordered, crystalline copper hydrides, atomically disordered, crystalline copper nitrates; atomically disordered, crystalline copper carbonates; atomically disordered, crystalline copper sulfides; atomically disordered, crystalline copper sulfadiazines; atomically disordered, crystalline copper acetates; atomically disordered, crystalline copper lactates; atomically disordered, crystalline copper citrates; atomically disordered, crystalline alkali copper thiosulphates (e.g., atomically disordered, crystalline sodium copper thiosulphate, atomically disordered, crystalline potassium copper thiosulphate)), atomically disordered, crystalline tin-containing materials (e.g., atomically disordered, crystalline tin; atomically disordered, crystalline tin alloys; atomically disordered, crystalline tin oxides; atomically disordered, crystalline tin hydroxides; atomically disordered, crystalline tin carbides; atomically disordered, crystalline tin nitrides; atomically disordered, crystalline tin borides; atomically disordered, crystalline tin sulfides; atomically disordered, crystalline tin myristates; atomically disordered, crystalline tin stearates; atomically disordered, crystalline tin oleates; atomically disordered, crystalline tin glutonates; atomically disordered, crystalline tin glutonates; atomically disordered, crystalline tin adipates; atomically disordered, crystalline tin silicates; atomically disordered, crystalline tin phosphides; atomically disordered, crystalline tin halides; atomically disordered, crystalline tin hydrides, atomically disordered, crystalline tin nitrates; atomically disordered, crystalline tin carbonates; atomically disordered, crystalline tin sulfides; atomically disordered, crystalline tin sulfadiazines; atomically disordered, crystalline tin acetates; atomically disordered, crystalline tin lactates; atomically disordered, crystalline tin citrates; atomically disordered, crystalline alkali tin thiosulphates (e.g., atomically disordered, crystalline sodium tin thiosulphate, atomically disordered, crystalline potassium tin thiosulphate)), atomically disordered, crystalline antimony-containing materials (e.g., atomically disordered, crystalline antimony; atomically disordered, crystalline antimony alloys; atomically disordered, crystalline antimony oxides; atomically disordered, crystalline antimony hydroxides; atomically disordered, crystalline antimony carbides; atomically disordered, crystalline antimony nitrides; atomically disordered, crystalline antimony borides; atomically disordered, crystalline antimony sulfides; atomically disordered, crystalline antimony myristates; atomically disordered, crystalline antimony stearates; atomically disordered, crystalline antimony oleates; atomically disordered, crystalline antimony glutonates; atomically disordered, crystalline antimony glutonates; atomically disordered, crystalline antimony adipates; atomically disordered, crystalline antimony silicates; atomically disordered, crystalline antimony phosphides; atomically disordered, crystalline antimony halides; atomically disordered, crystalline antimony hydrides, atomically disordered, crystalline antimony nitrates; atomically disordered, crystalline antimony carbonates; atomically disordered, crystalline antimony sulfides; atomically disordered, crystalline antimony sulfadiazines; atomically disordered, crystalline antimony acetates; atomically disordered, crystalline go antimony ld lactates; atomically disordered, crystalline antimony citrates; atomically disordered, crystalline alkali antimony thiosulphates (e.g., atomically disordered, crystalline sodium antimony thiosulphate, atomically disordered, crystalline potassium antimony thiosulphate)), atomically disordered, crystalline bismuth-containing materials (e.g., atomically disordered, crystalline bismuth; atomically disordered, crystalline bismuth alloys; atomically disordered, crystalline bismuth oxides; atomically disordered, crystalline bismuth hydroxides; atomically disordered, crystalline bismuth carbides; atomically disordered, crystalline bismuth nitrides; atomically disordered, crystalline bismuth borides; atomically disordered, crystalline bismuth sulfides; atomically disordered, crystalline bismuth myristates; atomically disordered, crystalline bismuth stearates; atomically disordered, crystalline bismuth oleates; atomically disordered, crystalline bismuth glutonates; atomically disordered, crystalline bismuth glutonates; atomically disordered, crystalline bismuth adipates; atomically disordered, crystalline bismuth silicates; atomically disordered, crystalline bismuth phosphides; atomically disordered, crystalline bismuth halides; atomically disordered, crystalline bismuth hydrides, atomically disordered, crystalline bismuth nitrates; atomically disordered, crystalline bismuth carbonates; atomically disordered, crystalline bismuth sulfides; atomically disordered, crystalline bismuth sulfadiazines; atomically disordered, crystalline bismuth acetates; atomically disordered, crystalline bismuth lactates; atomically disordered, crystalline bismuth citrates; atomically disordered, crystalline alkali bismuth thiosulphates (e.g., atomically disordered, crystalline sodium bismuth thiosulphate, atomically disordered, crystalline potassium bismuth thiosulphate)).

Forms of the Material and Methods of Applying the Material

In general, the metal-containing material can be in any desired form or formulation. For example, the material can be a coating on a substrate (e.g., in the form of a dressing, a coated medical implant), a free standing powder, a solution, or disposed within a pharmaceutically acceptable carrier.

In some embodiments, the metal-containing material can act as a preservative. In such embodiments, a form or formulation containing the metal-containing material can be prepared or without without additional preservatives. Moreover, in embodiments in which the metal-containing material acts as a preservative, the metal-containing material may be included in a therapeutic formulation containing other therapeutic agents (e.g., the metal-containing material may be included primarily in certain therapeutic compositions to act as a preservative).

Moreover, the material can be applied to the subject in any of a variety of ways, generally depending upon the form of the material as applied and/or the area of the condition to be treated. In general, the amount of material used is selected so that the desired therapeutic effect (e.g., reduction in the condition being treated) is achieved while the material introduces an acceptable level of toxicity (e.g., little or no toxicity) to the subject. Generally, the amount of the material used will vary with the conditions being treated, the stage of advancement of the condition, the age and type of host, and the type, concentration and form of the material as applied. Appropriate amounts in any given instance will be readily apparent to those skilled in the art or capable of determination by routine experimentation. In some embodiments, a single application of the material may be sufficient. In certain embodiments, the material may be applied repeatedly over a period of time, such as several times a day for a period of days, weeks, months or years.

Substrate Coatings

Examples of commercially available metal-containing materials include the Acticoat® family of dressings (Smith & Nephew, Hull, UK), which are formed of antimicrobial, anti-inflammatory atomically disordered, nanocrystalline silver-containing material coated on one or more substrates. Such dressings include the Acticoat® dressings, the Acticoat® dressings, the Acticoat® moisture coating dressings, and the Acticoat® absorbent dressings.

A coating of a metal-containing material (e.g., an antimicrobial, atomically disordered, nanocrystalline silver-containing material) can be formed on a substrate using a desired technique. In certain embodiments, the coating is formed by depositing the material on the substrate surface using chemical vapor deposition, physical vapor deposition, and/or liquid phase deposition. Exemplary deposition methods include vacuum evaporation deposition, arc evaporation deposition, sputter deposition, magnetron sputter deposition and ion plating.

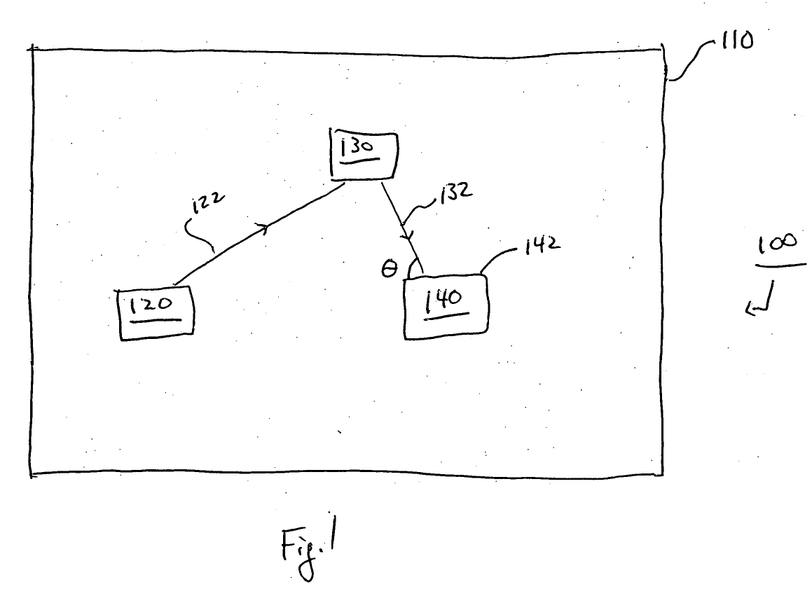

In some embodiments, the coating is prepared using physical vapor deposition. FIG. 1 shows a vapor deposition system 100 that includes a vacuum chamber 110, an energy source 120 (e.g., an electron beam source, an ion source, a laser beam, a magnetron source), a target 130 and a substrate 140. During operation, energy source 120 directs a beam of energy 122 to target 130, causing material 132 to be removed (e.g., by evaporation) from target 130 and directed to a surface 142 of substrate 140. At least a portion of the removed material 132 is deposited on surface 142.

In general, the values of the system parameters (e.g., the temperature of surface 142, the pressure of chamber 110, the angle of incidence of removed material 132 on surface 142, the distance between target 130 and surface 142) can be selected as desired. The temperature of surface 142 can be relatively low during the deposition process. For example, during the deposition process, the ratio of the temperature of substrate 140 to the melting point of the material forming target 130 (as determined in using Kelvin) can be about 0.5 or less (e.g., about 0.4 or less, about 0.35 or less, about 0.3 or less).

The pressure in chamber 110 can be relatively high. For example, vacuum evaporation deposition, electron beam deposition or arc evaporation, the pressure can be about 0.01 milliTorr or greater. For gas scattering evaporation (pressure plating) or reactive arc evaporation, the pressure in chamber 110 can be about 20 milliTorr or greater. For sputter deposition, the pressure in chamber 110 can be about 75 milliTorr or greater. For magnetron sputter deposition, the pressure in chamber 110 can be about 10 milliTorr or greater. For ion plating, the pressure in chamber 110 can be 200 milliTorr or greater.

The angle of incidence of removed material 132 on surface 142 (θ) can be relatively low. For example, the angle of incidence of removed material 132 on surface 142 can be about 75° or less (e.g., about 60° or less, about 45° or less, about 30° or less).

The distance between target 130 and surface 142 can be selected based upon the values of the other system parameters. For example, the distance between target 130 and surface 142 can be about 250 millimeters or less (e.g., about 150 millimeters or less, 125 millimeters or less, about 100 millimeters or less, about 90 millimeters or less, about 80 millimeters or less, about 70 millimeters or less, about 60 millimeters or less, about 50 millimeters or less, about 40 millimeters or less).

As noted above, it is believed that, the metal-containing material, when contacted with an alcohol or water-based electrolyte, can be released into the alcohol or water-based electrolyte (e.g., as ions, atoms, molecules and/or clusters). It is also believed that the ability to release the metal (e.g., as atoms, ions, molecules and/or clusters) on a sustainable basis from a coating is generally dependent upon a number of factors, including coating characteristics such as composition, structure, solubility and thickness, and the nature of the environment in which the device is used. As the level of atomic disorder is increased, it is believed that the amount of metal species released per unit time increases. For example, a silver metal film deposited by magnetron sputtering at a ratio of substrate temperature to the target melting point of less than about 0.5 and a working gas pressure of about 0.93 Pascals (about seven milliTorr) releases approximately ⅓ of the silver ions that a film deposited under similar conditions, but at four Pascals (about 30 milliTorr), will release over 10 days. Coatings formed with an intermediate structure (e.g., lower pressure, lower angle of incidence etc.) have been observed to have metal (e.g., silver) release values intermediate to these values as determined by bioassays. In general, to obtain relatively slow release of the metal, the coating should have a relatively low degree of atomic disorder, and, to obtain relatively fast release of the metal, the coating should have a relatively high degree of atomic disorder.

For continuous, uniform coatings, the time for total dissolution is generally a function of coating thickness and the nature of the environment to which the coating is exposed. The release of metal is believed to increase approximately linearly as the thickness of the coating is increased. For example, it has been observed that a two fold increase in coating thickness can result in about a two fold increase in longevity.

In certain embodiments, it is possible to manipulate the degree of atomic disorder, and therefore the metal release from a coating, by forming a thin film coating with a modulated structure. For example, a coating deposited by magnetron sputtering such that the working gas pressure was relatively low (e.g., about two Pascals or about 15 milliTorr) for about 50% of the deposition time and relatively high (e.g., about four Pascals or 30 milliTorr) for the remaining time, can result in a relatively rapid initial release of metal (e.g., ions, clusters, atoms, molecules), followed by a longer period of slow release. This type of coating is can be particularly effective on devices such as urinary catheters for which an initial rapid release is advantageous to achieve quick antimicrobial concentrations followed by a lower release rate to sustain the concentration of metal (e.g., ions, clusters, atoms, molecules) over a period of weeks.

It is further believed that the degree of atomic disorder of a coating can be manipulated by introducing one or more dissimilar materials into the coating. For example, one or more gases can be present in chamber 110 during the deposition process. Examples of such gases include oxygen-containing gases (e.g., oxygen, air, water), nitrogen-containing gases (e.g., nitrogen), hydrogen-containing gases (e.g., water, hydrogen), boron-containing gases (e.g., boron), sulfur-containing gases (e.g., sulfur), carbon-containing gases (e.g., carbon monoxide, carbon dioxide), silicon-containing gases, phosphorus-containing gases, and halogen-containing gases (e.g., fluorine, chlorine, bromine, iodine). The additional gas(es) can be co-deposited or reactively deposited with material 132. This can result in the deposition of an oxide, hydroxide, nitride, carbide, boride, sulfide, hydride, nitrate, carbonate, alkali thiosulphate (e.g., sodium thiosulphate, potassium thiosulphate), sulfadiazine, acetate, lactate, citrate, myristate, sorbate, stearate, oleate, glutonate, adipate, phosphide, silicate and/or halide material (e.g., an oxide of a metal-containing material, an oxide of a metal-containing material, a nitride of a metal-containing material, a carbide of a metal-containing material, a boride of a metal-containing material, a sulfide of a metal-containing material, a hydride of a metal-containing material, a halide of a metal-containing material, a nitrate of a metal-containing material, a carbonate of a metal-containing material, a sulfide of a metal-containing material, a sulfadiazine of a metal-containing material, a sulfadiazine of a metal-containing material, an acetate of a metal-containing material, a lactate of a metal-containing material, a citrate of a metal-containing material, a phosphide of a metal-containing material, a silicate of a metal-containing material, a myristate of a metal-containing material, a sorbate of a metal-containing material, a stearate of a metal-containing material, an oleate of a metal-containing material, a glutonate of a metal-containing material, an adipate of a metal-containing material, an alkali metal thiosulphate (e.g., sodium metal thiosulphate, potassium metal thiosulphate) of a metal-containing material). Without wishing to be bound by theory, it is believed that atoms and/or molecules of the additional gas(es) may become absorbed or trapped in the material, resulting in enhanced atomic disorder. The additional gas(es) may be continuously supplied during deposition, or may be pulsed to (e.g., for sequential deposition). In embodiments, the material formed can be constituted of a material with a ratio of material 132 to additional gas(es) of about 0.2 or greater. The presence of dissimilar atoms or molecules in the coating can enhance the degree of atomic disorder of the coating due to the difference in atomic radii of the dissimilar constituents in the coating.

The presence of dissimilar atoms or molecules in the coating may also be achieved by co-depositing or sequentially depositing one or more additional metal elements (e.g., one or more additional antimicrobial metal elements). Such additional metal elements include, for example, Au, Pt, Ta, Ti, Nb, Zn, V, Hf, Mo, Si, Al, and other transition metal elements. It is believed that the presence of dissimilar metal elements (one or more primary metal elements and one or more additional metal elements) in the coating can reduce atomic diffusion and stabilize the atomically disordered structure of the coating. A coating containing dissimilar metal elements can be formed, for example, using thin film deposition equipment with multiple targets. In some embodiments, sequentially deposited layers of the metal elements are discontinuous (e.g., islands within a the primary metal). In certain embodiments, the weight ratio of the additional metal(s) to the primary metal(s) is greater than about 0.2.

While FIG. 1 shows one embodiment of a deposition system, other embodiments are possible. For example, the deposition system can be designed such that during operation the substrate moves along rollers. Additionally or alternatively, the deposition system may contain multiple energy sources, multiple targets, and/or multiple substrates. The multiple energy sources, targets and/or substrates can be, for example, positioned in a line, can be staggered, or can be in an array.

In certain embodiments, two layers of the material are deposited on the substrate to achieve an optical interference effect. Alternatively, the two layers can be formed of different materials, with the outer (top) of the two layers being formed of an antimicrobial, atomically disordered, nanocrystalline silver-containing material, and the inner of the two layers having appropriate reflective properties so that the two layers can provide an interference effect (e.g., to monitor the thickness of the outer (top) of the two layers).

The substrate can be selected as desired. The substrate may be formed of one layer or multiple layers, which may be formed of the same or different materials.

In certain embodiments, the substrate can include one or more layers containing a bioabsorbable material. Bioabsorbable materials are disclosed, for example, in U.S. Pat. No. 5,423,859. In general, bioabsorbable materials can include natural bioabsorbable polymers, biosynethetic bioabsorbable polymers and synthetic bioabsorbable polymers. Examples of synthetic bioabsorbable polymers include polyesters and polylactones (e.g., polymers of polyglycolic acid, polymers of glycolide, polymers of lactic acid, polymers of lactide, polymers of dioxanone, polymers of trimethylene carbonate, polyanhydrides, polyesteramides, polyortheoesters, polyphosphazenes, and copolymers of the foregoing). Examples of natural bioabsorbable polymers include proteins (e.g., albumin, fibrin, collagen, elastin), polysaccharides (e.g., chitosan, alginates, hyaluronic acid). Examples of biosynthetic polymers include polyesters (e.g., 3-hydroxybutyrate polymers).

In some embodiments, the substrate includes multiple layers (e.g., two layers, three layers, four layers, five layers, six layers, seven layers, eight layers, nine layers, 10 layers). The layers can be laminated together (e.g., by thermal fusing, stitching and/or ultrasonic welding).

One or more layers (e.g., an outer layer) of a multi-layer substrate can be formed of a perforated (and optionally non-adherent) material (e.g., a woven material or a non-woven material) that can allow fluid to penetrate or diffuse therethrough. Such materials include, for example, cotton, gauze, polymeric nets (e.g., polyethylene nets, nylon nets, polypropylene nets, polyester nets, polyurethane nets, polybutadiene nets), polymeric meshes (e.g., polyethylene meshes, nylon meshes, polypropylene meshes, polyester meshes, polyurethane meshes, polybutadiene meshes) and foams (e.g., an open cell polyurethane foam). Examples of commercially available materials include DELNET™ P530 non-woven polyethylene veil (Applied Extrusion Technologies, Inc., Middletown, Del.), Exu-Dry CONFORMANT2™ non-woven polyethylene veil (Frass Survival Systems, Inc., NY, N.Y.), CARELLE™ material (Carolina Formed Fabrics Corp.), NYLON90™ material (Carolina Formed Fabrics Corp.), N-TERFACE™ material (Winfield Laboratories, Inc., Richardson, Tex.), HYPOL™ hydrophilic polyurethane foam (W. R. Grace & Co., NY, N.Y.).

One or more layers (e.g., an inner layer) of a multi-layer substrate can be formed of an absorbent material (e.g., a woven material or a non-woven material) formed of, for example, rayon, polyester, a rayon/polyester blend, polyester/cotton, cotton and/or cellulosic fibers. Examples include creped cellulose wadding, air felt, air laid pulp fibers and gauze. An example of a commercially available material is SONATRA™ 8411 70/30 rayon/polyester blend (Dupont Canada, Mississauga, Ontario).

One or more layers (e.g., an outer layer) of a multi-layer substrate can be formed of an occlusive or semi-occlusive material, such as an adhesive tape or polyurethane film (e.g., to secure the device to the skin and/or to retain moisture).

In some embodiments, the layers in a multi-layer substrate are laminated together (e.g., at intermittent spaced locations) by ultrasonic welds. Typically, heat (e.g., generated ultrasonically) and pressure are applied to either side of the substrate at localized spots through an ultrasonic horn so as to cause flowing of at least one of the plastic materials in the first and second layers and the subsequent bonding together of the layers on cooling. The welds can be formed as localized spots (e.g., circular spots). The spots can have a diameter of about 0.5 centimeter or less.

The shape of the substrate can generally be varied as desired. For example, the substrate can be in the shape of a film, a fiber or a powder.

The substrate/coating article can be used in a variety of articles. For example, the article can be in the shape of a medical device. Exemplary medical devices include wound closure devices (e.g., sutures, staples, adhesives), tissue repair devices (e.g., meshes, such as meshes for hernia repair), prosthetic devices (e.g., internal bone fixation devices, physical barriers for guided bone regeneration, stents, valves, electrodes), tissue engineering devices (e.g., for use with a blood vessel, skin, a bone, cartilage, a liver), controlled drug delivery systems (e.g., microcapsules, ion-exchange resins) and wound coverings and/or fillers (e.g., alginate dressings, chitosan powders). In some embodiments, the article is a transcutaneous medical device (e.g., a catheter, a pin, an implant), which can include the substrate/coating supported on, for example, a solid material (e.g., a metal, an alloy, latex, nylon, silicone, polyester and/or polyurethane). In some embodiments, the article is in the form of a patch (e.g., a patch having an adhesive layer for adhering to the skin, such as a transdermal patch).

Subsequent to deposition, the material can optionally be annealed. In general, the anneal is conducted under conditions to increase the stability (e.g., shelf life) of the material while maintaining the desired therapeutic activity of the material. In certain embodiments, the material can be annealed at a temperature of about 200° C. or less (e.g., about room temperature).

The substrate/coating is typically sterilized prior to use (e.g., without applying sufficient thermal energy to anneal out the atomic disorder). The energy used for sterilization can be, for example, gamma radiation or electron beam radiation. In some embodiments, ethylene oxide sterilization techniques are used to sterilize the substrate/coating.

Free Standing Powders

A free standing powder can be prepared by, for example, cold working or compressing to impart atomic disorder to the powder. In certain embodiments, a free standing powder is prepared by forming a coating of the material as described above, and then removing the material from the surface of the substrate. For example, the material can be scraped from the surface of the substrate by one or more scrapers. In embodiments in which the substrate moves during deposition of the material, the scrapers can remove the material as the substrate moves. The scrapers can be, for example, suspended above the substrate. Such scrapers can be, for example, weighted and/or spring loaded to apply pressure sufficient to remove the material as the substrate moves. In some embodiments (e.g., when a continuous belt is used), the scrapers can be located above the end rollers to remove the material with a reverse dragging action as the substrate rounds the end roller.

A free standing powder can be used to treat a condition in various ways. As an example, the powder can sprinkled onto the subject's skin. As another example, the powder can be inhaled using an inhaler, such as a dry powder inhaler. In some embodiments, a dry powder can be in the form of an aerosol, which contains, for example, at least about 10 (e.g., at least about 20, at least about 30) weight percent and/or at most about 99 (e.g., at most about 90, at most about 80, at most about 70, at most about 60, at most about 50) weight percent of the dry powder.

In certain embodiments (e.g., when the free standing powder is inhaled), the average particle size of the free standing powder is selected to reduce the likelihood of adverse reaction(s) of the particles in the tissue and/or to deposit the powder onto specific anatomical locations (e.g., tissue contacted by the free standing powder during inhalation). In some embodiments, the average particle size is selected (e.g., less than about 10 microns) so that a relatively small amount of the particles get into the lower respiratory tract. In embodiments, a free standing powder can have an average particle size of less than about 10 microns (e.g., less than about eight microns, less than about five microns, less than about two microns, less than about one micron, less than about 0.5 micron) and/or at least about 0.01 micron (e.g., at least about 0.1 micron, at least about 0.5 micron).

Powder Impregnated Materials

The metal-containing material can be in the form of a powder impregnated material. Such powder impregnated materials can, for example, be in the form of a hydrocolloid having the free standing powder blended therein. A powder impregnated material can be, for example, in the form of a dressing, such as a hydrocolloid dressing.

Solutions

The metal-containing material can be in the form of a solution (e.g., a solvent-based solution). The solution can be formed, for example, by dissolving a free standing powder of the material in a solvent for the powder. As an example, a container (e.g., a tea bag-type container) with the free standing powder within it can be immersed in the water or solvent. As another example, a substrate (e.g., in the form of a strip or a bandage) carrying the material can be immersed in the solvent. In certain embodiments, it can be preferable to form a solution by dissolving a free standing powder of the metal-containing material in a solvent because this can be a relatively convenient approach to forming a solution. A solution also refers to a suspension that contains one or more metal-containing materials. As an example, a suspension can be formed by dissolving a metal-containing material (e.g., a nanocrystalline silver-containing material) in a liquid (e.g., water) for a period of time (e.g., several days) so that particles of the metal-containing material are suspended (e.g., by Brownian motion) in the liquid. In some embodiments, a suspended particle of metal-containing material can have, for example, a diameter of the order of from about 10 nanometers to about 20 nanometers. A solution also includes a dispersion.

In certain embodiments, the solution containing the metal-containing material is contacted with the subject relatively soon after formation of the solution. For example, the solution containing the metal-containing material can be contacted with the subject within about one minute or less (e.g., within about 30 seconds or less, within about 10 seconds or less) of forming the solution containing the metal-containing material. In some embodiments, a longer period of time lapses before the solution containing the metal-containing material is contacted with the subject. For example a period of time of at least about 1.5 minutes (e.g., at least about five minutes, at least about 10 minutes, at least about 30 minutes, at least about one hour, at least about 10 hours, at least about a day, at least about a week) lapses between the time the solution containing the metal-containing material is formed and the solution containing the metal-containing material is contacted with the subject.

In some embodiments, lowering the pH of the solution (e.g., to less than about 6.5, such as from about 3.5 to about 6.5) can allow for a higher concentration of the dissolved material and/or a faster rate of dissolution. The pH of the solution can be lowered, for example, by adding acid to the solution (e.g., by adding CO₂ to the solution to form carbonic acid).

A solution containing the metal-containing material can be contacted with the subject with or without the use of a device. As an example, a solution containing the metal-containing material can be contacted with the skin, mouth, ears or eyes as a rinse, a bath, a wash, a gargle, a spray and/or drops. As another example, the solution can be injected using a small needle injector and/or a needleless injector. As an additional example, a solution containing the metal-containing material can be formed into an aerosol (e.g., an aerosol prepared by a mechanical mister, such as a spray bottle or a nebulizer), and the aerosol can be contacted with the subject using an appropriate device (e.g., a hand held inhaler, a mechanical mister, a spray bottle, a nebulizer, an oxygen tent). As a further example, a solution containing the metal-containing material can be contacted with the second area via a catheter.

In embodiments in which onychomycosis is being treated, the method can include first hydrating the nail with urea (1-40%) or lactic acid (10-15%), followed by treatment with the metal-containing material, which may contain an appropriate solvent (e.g., DMSO) for penetration through the nail. Alternatively or additionally, onychomycosis can be treated by injecting (e.g., via a needleless injector and/or a needle) the metal-containing material to the affected area.

Typically, the solvent is a relatively hydrophilic solvent. Examples of solvents include water, DMSO and alcohols. In certain embodiments, a water-based solution is a buffered solution. In some embodiments, a water-based solution contains carbonated water. In embodiments, more than one solvent can be used.

In some embodiments, the solution can contain about 0.001 weight percent or more (e.g., about 0.01 weight percent or more, about 0.02 weight percent or more, about 0.05 weight percent or more, about 0.1 weight percent or more, about 0.2 weight percent or more, about 0.5 weight percent or more, about one weight percent or more) of the metal-containing material and/or about 10 weight percent or less (e.g., about five weight percent or less, about four weight percent or less, about three weight percent or less, about two weight percent or less, about one weight percent or less) of the metal-containing material. As an example, in certain embodiments in which respiratory conditions are being treated, a solution can contain at least about 0.001 (e.g., at least about 0.01, at least about 0.02, at least about 0.05, at least, about 0.1) weight percent and/or at most about 0.5 (e.g. at most about 0.4, at most about 0.3) weight percent of the metal-containing material

Pharmaceutical Carrier Compositions

The metal-containing material can disposed (e.g., suspended) within a pharmaceutically acceptable carrier. The formulation can be, for example, a semi-solid, a water-based hydrocolloid, an oil-in-water emulsion, a water-in-oil emulsion, a non-dried gel, and/or a dried gel. Typically, when disposed in a pharmaceutically acceptable carrier, the metal-containing material is applied to the skin.

Examples of pharmaceutically acceptable carriers include creams, ointments, gels, sprays, solutions, drops, powders, lotions, pastes, foams and liposomes.

The formulation can contain about 0.01 weight percent or more (e.g., about 0.1 weight percent or more, about 0.5 weight percent or more, about 0.75 weight percent or more, about one weight percent or more, about two weight percent or more, about five weight percent or more, about 10 weight percent or more) of the metal-containing material and/or about 50 weight percent or less (e.g., about 40 weight percent or less, about 30 weight percent or less, about 20 weight percent or less, about 20 weight percent or less, about 15 weight percent or less, about 10 weight percent or less, about five weight percent or less) of the metal-containing material.

Formulations can optionally include one or more components which can be biologically active or biologically inactive. Examples of such optional components include base components (e.g., water and/or an oil, such as liquid paraffin, vegetable oil, peanut oil, castor oil, cocoa butter), thickening agents (aluminum stearate, hydrogen lanolin), gelling agents, stabilizing agents, emulsifying agents, dispersing agents, suspending agents, thickening agents, coloring agents, perfumes, excipients (starch, tragacanth, cellulose derivatives, polyethylene glycols, silicones, bentonites, silicic acid, talc), foaming agents (e.g., surfactants), surface active agents, preservatives (e.g., methyl paraben, propyl paraben) and cytoconductive agents (e.g., betaglucan). In some embodiments, a formulation includes petrolatum. In certain embodiments, a pharmaceutical carrier composition can include a constituent (e.g., DMSO) to assist in the penetration of skin.

While the foregoing has described embodiments in which a single condition is treated, in some embodiments multiple conditions can be treated. The multiple conditions can be the same type of condition (e.g., multiple skin conditions) or different types of conditions. For example, a dressing formed of one or more substrates coated with an appropriate metal-containing material (e.g., antimicrobial, atomically disordered, silver-containing material) can be applied to an area of the skin having multiple skin conditions (e.g., a bum and psoriasis) so that the metal-containing material treats the multiple skin conditions.

Moreover, while the foregoing has described embodiments that involve one method of contacting a subject with the metal-containing material, in other embodiments, more than one method of contacting a subject with the metal-containing material can be used. For example, the methods can include one or more of ingestion (e.g., oral ingestion), injection (e.g., using a needle, using a needleless injector), topical administration, inhalation (e.g., inhalation of a dry powder, inhalation of an aerosol) and/or application of a dressing.

Furthermore, while the foregoing has described embodiments in which one form of the metal-containing material is used, in other embodiments, more than one form of the metal-containing material can be used. For example, the methods can include using the metal-containing material in the form of a coating (e.g., a dressing), a free standing powder, a solution and/or a pharmaceutical carrier composition.

In general, the form of the metal-containing material can be selected as desired. Typically, the form of the metal-containing material can be selected based, at least in part, upon the area of the subject to be contacted with the metal-containing material. In certain embodiments, the metal-containing material can be effectively used in the oral cavity when in the form of a swab, a foam or a sponge that is used to wipe the oral cavity. In some embodiments, the metal-containing material can be effectively used in the oral cavity when in the form of a solution that is rinsed or gargled. In certain embodiments, the metal-containing material can be effectively used when in the form of an article (e.g., a tape, a pill, a capsule, a tablet, a suppository or lozenge) As an example, an article containing a metal-containing material can be used in the oral cavity (e.g., a tape, a pill, a capsule, a tablet or a lozenge) to treat a condition by allowing the subject to, for example, suck the article. As another example, an article containing a metal-containing material can be used for anal application (e.g., a suppository) to treat a condition (e.g., a gastrointestinal condition, such as lower gastrointestinal condition). In some embodiments, the article can be a sustained release article (e.g., a sustained release capsule) which can allow the metal-containing material to be released at a predetermined rate (e.g., a relatively constant rate). In some embodiments, an article can include a material (e.g., in the form of a coating and/or in the form of a matrix material) that allows the article to pass through certain portions of the gastrointestinal system with relatively little (e.g., no) release of the metal-containing material, but that allows a relatively large amount of the metal-containing material to be released in a desired portion of the gastrointestinal system. As an example,, the article can be an enteric article (e.g., an enteric coated tablet) so that the article to passes through the stomach with little (e.g., no) metal-containing material being released, and so that the metal-containing material is relatively easily released by the article in the intestines. In some embodiments, the metal-containing material can be effectively used in the nasal cavity when in the form of a mist (e.g., a nebulized mist) that is inhaled. In certain embodiments, the metal-containing material is effectively used in the nasal cavity when in the form of a dry powder that is inhaled (e.g., via a dry powder inhaler). In certain embodiments, the metal-containing material can be effectively used on the skin when in the form of a coating on a substrate (e.g., in the form of a dressing), a powder impregnated material, a solution (e.g., sprayed onto the skin), a pharmaceutical carrier composition (e.g., topically applied) or a free standing powder (e.g., sprinkled on the skin).

Generally, the size of the area contacted with the metal-containing material can be selected as desired. For example, the size of the area contacted with the metal-containing material can be about one square millimeter or larger (e.g., about 10 square millimeters or larger, about 50 square millimeters or larger, about one square centimeter or larger, about 10 square centimeters or larger, about 25 square centimeters or larger, about 50 square centimeters or larger, 100 square centimeters or larger, about 250 square centimeters or larger, about 375 square centimeters or larger, about 500 square centimeters or larger).

In embodiments, the distance between the area of the subject susceptible to the condition and the area of the subject contacted with the metal-containing material can be, for example, at least about 0.1 centimeter (e.g., at least about 0.5 centimeter, at least about one centimeter, at least about two centimeters, at least about three centimeters, at least about four centimeters, at least about five centimeters, at least about 10 centimeters, at least about 25 centimeters, at least about 50 centimeters, at least about 75 centimeters, at least about one meter, at least about 1.1 meters, at least about 1.2 meters, at least about 1.3 meters, at least about 1.4 meters, at least about 1.5 meters) and/or about 10 meters or less (e.g., about five meters or less, about four meters or less, about three meters or less, about two meters or less, about one meter or less, about 0.5 meter or less, about 0.1 meter or less).

In some embodiments, the area of the subject susceptible to the condition and the area of the subject contacted with the metal-containing material are different areas on the same portion of the subject. The area of the subject susceptible to the condition can be one area of the subject's skin, and the area of the subject contacted with the metal-containing material can be a different area of the subject's skin. The area of the subject susceptible to the condition can be one area of the subject's oral cavity, and the area of the subject contacted with the metal-containing material can be a different area of the person's oral cavity. The area of the subject susceptible to the condition can be one area of the person's nasal cavity, and the area of the subject contacted with the metal-containing material can be a different area of the person's nasal cavity. The area of the subject susceptible to the condition can be one area of the subject's lungs, and the area of the subject contacted with the metal-containing material can be a different area of one of the subject's lungs. The area of the subject susceptible to the condition can be one of the subject's bones, one of the subject's joints, and the area of the subject contacted with the metal-containing material can be a different area of one of the subject's joints. The area of the subject susceptible to the condition can be one of the subject's joints, one of the subject's joints, and the area of the subject contacted with the metal-containing material can be a different area of one of the subject's joints. The area of the subject susceptible to the condition can be one area of one of the subject's muscles, and the area of the subject contacted with the metal-containing material can be a different area of one of the subject's muscles. The area of the subject susceptible to the condition can be one area of one of the subject's tendons, and the area of the subject contacted with the metal-containing material can be a different area of one of the subject's tendons. The area of the subject susceptible to the condition can be one area of the subject's heart, and the area of the subject contacted with the metal-containing material can be a different area of the subject's heart. The area of the subject susceptible to the condition can be one area of the subject's lymphatic system, and the area of the subject contacted with the metal-containing material can be a different area of the subject's lymphatic system. The area of the subject susceptible to the condition can be a first area of one of the subject's blood vessels (e.g., a vein or an artery), and the area of the subject contacted with the metal-containing material can be a area of one of the subject's blood vessels.

In certain embodiments, the area of the subject susceptible to the condition and the area of the subject contacted with the metal-containing material are different portions of the subject. The area of the subject susceptible to the condition can be a portion of the subject's skin, and the area of the subject contacted with the metal-containing material can be a portion of the subject's respiratory system, a portion of the subject's musculo-skeletal system, a portion of the subject's gastrointestinal system, a portion of the subject's circulatory system, a portion of the subject's sublingual area, or a portion of the subject's subdermal area. The area of the subject susceptible to the condition can be a portion of the subject's respiratory system, and the area of the subject contacted with the metal-containing material can be a portion of the subject's skin, a portion of the subject's musculo-skeletal system, a portion of the subject's gastrointestinal system, a portion of the subject's circulatory system, a portion of the subject's sublingual area, or a portion of the subject's subdermal area. The area of the subject susceptible to the condition can be a portion of the subject's musculo-skeletal system, and the area of the subject contacted with the metal-containing material can be a portion of the subject's skin, a portion of the subject's respiratory system, a portion of the subject's gastrointestinal system, a portion of the subject's circulatory system, a portion of the subject's sublingual area, or a portion of the subject's subdermal area. The area of the subject susceptible to the condition can be a portion of the subject's circulatory system, and the area of the subject contacted with the metal-containing material can be a portion of the subject's skin, a portion of the subject's respiratory system, a portion of the subject's musculo-skeletal system, a portion of the subject's gastrointestinal system, a portion of the subject's sublingual area, or a portion of the subject's subdermal area. The area of the subject susceptible to the condition can be a portion of the subject's gastrointestinal system, and the area of the subject contacted with the metal-containing material can be a portion of the subject's he subject's skin, a portion of the subject's respiratory system, a portion of the subject's musculo-skeletal system, a portion of the subject's circulatory system, a portion of the subject's sublingual area, or a portion of the subject's subdermal area. The area of the subject susceptible to the condition can be a portion of the subject's sublingual area, and the area of the subject contacted with the metal-containing material can be a portion of the subject's skin, a portion of the subject's respiratory system, a portion of the subject's gastrointestinal system, a portion of the subject's circulatory system, a portion of the subject's musculo-skeletal system, or a portion of the subject's subdermal area. The area of the subject susceptible to the condition can be a portion of the subject's subdermal area, and the area of the subject contacted with the metal-containing material can be a portion of the subject's skin, a portion of the subject's respiratory system, a portion of the subject's gastrointestinal system, a portion of the subject's circulatory system, a portion of the subject's musculo-skeletal system, or a portion of the subject's sublingual area.

In some embodiments, more than one area of the subject susceptible to the condition can be treated. The multiple treated areas of the subject can be different portions of the same type of body system (e.g., multiple portions of the subject's skin, multiple portions of the subject's respiratory system, multiple portions of the subject's musculo-skeletal system, multiple portions of the subject's circulatory system, or multiple portions of the subject's gastrointestinal system), or the multiple treated areas of the subject can be portions of different types of body systems (e.g., a portion of the subject's skin, and one or more portions of the subject's respiratory system, one or more portions of the subject's musculo-skeletal system, one or more portions of the subject's circulatory system, and/or one or more portions of the subject's gastrointestinal system; a portion of the subject's respiratory system, and one or more portions of the subject's skin, one or more portions of the subject's musculo-skeletal system, one or more portions of the subject's circulatory system, and/or one or more portions of the subject's gastrointestinal system; a portion of the subject's musculo-skeletal system, and one or more portions of the subject's skin, one or more portions of the subject's respiratory system, one or more portions of the subject's circulatory system, and/or one or more portions of the subject's gastrointestinal system; a portion of the subject's circulatory system, and one or more portions of the subject's skin, one or more portions of the subject's respiratory system, one or more portions of the subject's musculo-skeletal system, and/or one or more portions of the subject's gastrointestinal system; a portion of the subject's gastroinstestinal system, and one or more portions of the subject's skin, one or more portions of the subject's respiratory system, one or more portions of the subject's musculo-skeletal system, and/or one or more portions of the subject's circulatory system).

In certain embodiments, more than one condition of the subject can be prophylactically treated. The multiple prophylactically treated conditions can be conditions of the same type (e.g., multiple bacterial conditions, multiple microbial conditions, multiple inflammatory conditions, multiple fungal conditions, multiple viral conditions, or multiple cancerous conditions), or the multiple treated conditions can be conditions of different types (e.g., a bacterial condition, and one or more microbial conditions, one or more bacterial conditions, one or more inflammatory conditions, one or more fungal conditions, one or more viral conditions, and/or one or more cancerous conditions; a microbial condition, and one or more bacterial conditions, one or more inflammatory conditions, one or more fungal conditions, one or more viral conditions, and/or one or more cancerous conditions; an inflammatory condition, and one or more bacterial conditions, one or more microbial conditions, one or more fungal conditions, one or more viral conditions, and/or one or more cancerous conditions; a fungal condition, and one or more inflammatory conditions, one or more microbial conditions, one or more viral conditions, and/or one or more cancerous conditions; a viral condition, and one or more bacterial conditions, one or more inflammatory conditions, one or more fungal conditions, one or more microbial conditions, and/or one or more cancerous conditions; a cancerous condition, and one or more bacterial conditions, one or more inflammatory conditions, one or more fungal conditions, one or more viral conditions, and/or one or more microbial conditions). The multiple treated conditions can be conditions of the same type of body system (e.g., multiple skin conditions, multiple integument conditions, multiple respiratory conditions, multiple musculo-skeletal conditions, multiple circulatory conditions, multiple mucosal conditions, multiple serosal conditions), or the multiple treated conditions can be conditions of different types of body systems (a skin condition, and one or more respiratory conditions, one or more musculo-skeletal conditions, one or more circulatory conditions, and/or one or more mucosal or serosal conditions; a respiratory condition, and one or more skin conditions, one or more musculo-skeletal conditions, one or more circulatory conditions, and/or one or more mucosal or serosal conditions; a musculo-skeletal condition, and one or more respiratory conditions, one or more skin conditions, one or more circulatory conditions, and/or one or more mucosal or serosal conditions; a circulatory condition, and one or more respiratory conditions, one or more musculo-skeletal conditions, one or more skin conditions, and/or one or more mucosal or serosal conditions; a mucosal or serosal condition, and one or more-respiratory conditions, one or more musculo-skeletal conditions, one or more circulatory conditions, and/or one or more skin conditions).

In some embodiments, more than one area of the subject can be contacted with the metal-containing material. The multiple contacted areas of the subject can be different portions of the same type of body system (e.g., multiple portions of the subject's skin, multiple portions of the subject's respiratory system, multiple portions of the subject's musculo-skeletal system, multiple portions of the subject's circulatory system, or multiple portions of the subject's gastrointestinal system), or the multiple contacted areas of the subject can be portions of different types of body systems (e.g., a portion of the subject's skin, and one or more portions of the subject's respiratory system, one or more portions of the subject's musculo-skeletal system, one or more portions of the subject's circulatory system, and/or one or more portions of the subject's gastrointestinal system; a portion of the subject's respiratory system, and one or more portions of the subject's skin, one or more portions of the subject's musculo-skeletal system, one or more portions of the subject's circulatory system, and/or one or more portions of the subject's gastrointestinal system; a portion of the subject's musculo-skeletal system, and one or more portions of the subject's skin, one or more portions of the subject's respiratory system, one or more portions of the subject's circulatory system, and/or one or more portions of the subject's gastrointestinal system; a portion of the subject's circulatory system, and one or more portions of the subject's skin, one or more portions of the subject's respiratory system, one or more portions of the subject's musculo-skeletal system, and/or one or more portions of the subject's gastrointestinal system; a portion of the subject's gastroinstestinal system, and one or more portions of the subject's skin, one or more portions of the subject's respiratory system, one or more portions of the subject's musculo-skeletal system, and/or one or more portions of the subject's circulatory system).

In certain embodiments, more than one metal-containing material can be used to prophylactically treat the condition of the subject. The multiple metal-containing material s can each contain at least one common metal (e.g., multiple silver-containing materials, multiple gold-containing materials, multiple platinum-containing materials, multiple palladium-containing materials, multiple iridium-containing materials, multiple zinc-containing materials, multiple copper-containing materials, multiple tin-containing materials, multiple antimony-containing materials, and/or multiple bismuth-containing materials), or the multiple metal-containing materials can contain no common metal elements (e.g., only one silver-containing material, only one gold-containing material, only one platinum-containing material, only one palladium-containing material, only one iridium-containing material, only one zinc-containing material, only one copper-containing material, only one tin-containing material, only one antimony-containing material, and/or only one bismuth-containing material). One or more of the multiple metal-containing materials can be an antimicrobial material, a disordered crystalline material, and/or a nanocrystalline material. One or more of the metal-containing materials can be an antimicrobial, atomically disordered, nanocrystalline crystalline material.

In certain embodiments, one or more areas (e.g., multiple areas) of a subject can be contacted with one or more metal-containing materials (e.g., multiple metal-containing materials) to prophylactically treat one or more conditions (e.g., multiple conditions) of the subject located at one or more areas (e.g., multiple areas) of the subject.

In some embodiments, the area of the subject susceptible to the condition and the area of the subject contacted with the metal-containing material may be different portions of a subject (e.g., different portions of the subject's skin, different portions of the subject's respiratory system, different portions of the subject's circulatory system, different portions of the subject's gastrointestinal system, different portions of the subject's musculo-skeletal system) that have the condition. As an example, the first and second areas can be different portions of a subject's skin (e.g., different portions of the skin on a subject's arm, different portions of the skin on a subject's leg, different portions of the skin on a subject's torso, different portions of the skin on a subject's neck, different portions of the skin on a subject's head) that have a skin condition (e.g., a burn, eczema, atopic eczema, acrodermatitis continua, contact allergic dermatitis, contact irritant dermatitis, dyshidrotic eczema, pompholyx, lichen simplex chronicus, nummular eczema, seborrheic dermatitis, stasis eczema, erythroderma, an insect bite, mycosis fungoides, pyoderma gangrenosum, eythrema multiforme, rosacea, onychomycosis, acne, acne vulgaris, neonatal acne, infantile acne, pomade acne, psoriasis, Reiter's syndrome, pityriasis rubra pilaris, hyperpigmentation, vitiligo, scarring conditions, and hyperproliferative variants of the disorders of keratinization).

As explained above, the metal-containing materials can be in any of a variety of forms when delivered to a subject (e.g., free standing powders, solutions, creams, ointments, gels, sprays, solutions, drops, powders, lotions, pastes, foams, liposomes, coatings on a substrate), and the metal-containing materials can be delivered to a subject in a variety of ways, including, for example, ingestion (e.g., oral ingestion), injection (e.g., using a needle, using a needleless injector), topical administration, inhalation (e.g., inhalation of a dry powder, inhalation of an aerosol) or application of a dressing. As also explained above, various subjects, conditions, areas of conditions, metal-containing materials, forms of metal-containing materials, areas for applying metal-containing materials, and methods of delivering metal-containing materials can be used.

Moreover, the metal-containing material can be used in various industrial applications. For example, the metal-containing material can be used to reduce and/or prevent microbial growth on industrial surfaces (e.g., industrial surfaces where microbial growth may occur, such as warm and/or moist surfaces). Examples of industrial surfaces include heating pipes and furnace filters. In certain embodiments, the metal-containing material can be disposed (e.g., coated or sprayed) on the surface of interest to reduce and/or prevent microbial growth. This can be advantageous in preventing the spread of microbes via, for example, heating and/or air circulation systems within buildings.

Furthermore, while methods have been described in which an area of a subject is prophylactically treated by contacting the same or a different area of the subject with a metal-containing material, in some embodiments, a subject is prophylactically treated by contacting (e.g., by washing, by rinsing, by coating, such as vapor deposited coating) an object other than the subject with a metal-containing material. In general, the object can be any object that is contacted with the subject or is used to deliver a material (e.g., a therapeutic agent, such as a drug) to the subject. In some embodiments, the object can be a medical device, a mechanical misters, a spray bottle, a nebulizer, an oxygen tent, a dry powder inhaler, a needle, a needleless injector, a dressing, a solution dropper, a container for a solution (e.g., to allow for gargling or washing). Examples of such objects are noted above. Additional examples of objects include medical instruments (e.g., minimally invasive medical instruments, such as laparoscopic instruments), respiratory equipment and catheters. Examples of medical instruments include scalpels, retractors, clamps, colonoscopes, endoscopes, trocars, grabbers, pushers and cutters. Examples of respiratory equipment includes equipment for subject intubation and/or treating certain conditions, such as cystic fibrosis. Examples of such equipment include tracheal tubes, CPAP tubes, Y tubes, conducting tubes, conducting ports, connectors, nebulizers, oxygen suppliers, nose pieces, mouth pieces). Examples of catheters include urinary catheters, indwelling catheters and Foley catheters.

The following examples are illustrative and not intended as limiting.

### EXAMPLE I

An adult male human subject without swimmer's ear is prophylactically treated for swimmer's ear as follows. Foam ear plugs containing antimicrobial, atomically disordered, nanocrystalline-silver containing material are prepared. The subject inserts the foam ear plugs in his ears during swimming.

### EXAMPLE II

An adult male human subject without swimmer's ear is prophylactically treated for swimmer's ear as follows. Foam ear plugs containing antimicrobial, atomically disordered, nanocrystalline-silver containing material are prepared. The subject inserts the foam ear plugs in his ears after swimming.

### EXAMPLE III

An adult male human subject without nosocomial pneumonia is prophylactically treated for nosocomial pneumonia during a hospital stay as follows. A swab containing antimicrobial, atomically disordered, nanocrystalline-silver containing material is prepared. The swab is inserted into the subject's oral cavity and swept through the oral cavity. This procedure is repeated three times a day during the duration of the hospital stay.

### EXAMPLE IV

An adult male human subject without nosocomial pneumonia is prophylactically treated for nosocomial pneumonia during a hospital stay as follows. A lozenge containing antimicrobial, atomically disordered, nanocrystalline-silver containing material is prepared. The lozenge is inserted into the subject's oral cavity and sucked for 15 minutes. This procedure is repeated three times a day during the duration of the hospital stay.

### EXAMPLE V

An adult male human subject without nosocomial pneumonia is prophylactically treated for nosocomial pneumonia during a hospital stay as follows. A sponge containing antimicrobial, atomically disordered, nanocrystalline-silver containing material is prepared. The sponge is inserted into the subject's oral cavity and swept through the oral cavity. This procedure is repeated three times a day during the duration of the hospital stay.

### EXAMPLE VI

An adult male human subject without nosocomial pneumonia is prophylactically treated for nosocomial pneumonia during a hospital stay as follows. A foam article containing antimicrobial, atomically disordered, nanocrystalline-silver containing material is prepared. The foam article is inserted into the subject's oral cavity and swept through the oral cavity. This procedure is repeated three times a day during the duration of the hospital stay.

### EXAMPLE VI

An adult male human subject without nosocomial pneumonia is prophylactically treated for nosocomial pneumonia during a hospital stay as follows. A tape containing antimicrobial, atomically disordered, nanocrystalline-silver containing material is prepared. The tape is inserted into the subject's oral cavity and kept in the oral cavity for 15 minutes. This procedure is repeated three times a day during the duration of the hospital stay.

### EXAMPLE VIII

An adult male human subject without nosocomial pneumonia is prophylactically treated for nosocomial pneumonia during a hospital stay as follows. A solution containing antimicrobial, atomically disordered, nanocrystalline-silver containing material is prepared. The subject rinses his oral cavity with the solution. This procedure is repeated three times a day during the duration of the hospital stay.

### EXAMPLE IX

An adult male human subject without nosocomial pneumonia is prophylactically treated for nosocomial pneumonia during a hospital stay as follows. A solution containing antimicrobial, atomically disordered, nanocrystalline-silver containing material is prepared. The subject gargles the solution. This procedure is repeated three times a day during the duration of the hospital stay.

### EXAMPLE X

An adult male human subject without nosocomial pneumonia is prophylactically treated for nosocomial pneumonia during a hospital stay as follows. A solution containing antimicrobial, atomically disordered, nanocrystalline-silver containing material is prepared. The solution is formed into a mist, and the subject inhales the mist through his oral cavity. This procedure is repeated three times a day during the duration of the hospital stay.

### EXAMPLE XI

An adult male human subject without nosocomial pneumonia is prophylactically treated for nosocomial pneumonia during a hospital stay as follows. A solution containing antimicrobial, atomically disordered, nanocrystalline-silver containing material is prepared. The solution is formed into a mist, and the subject inhales the mist through his nasal cavity. This procedure is repeated three times a day during the duration of the hospital stay.

### EXAMPLE XII

An adult male human subject without ventilator-associated pneumonia is prophylactically treated for ventilator-associated pneumonia during a hospital stay as follows. A swab containing antimicrobial, atomically disordered, nanocrystalline-silver containing material is prepared. The swab is inserted into the subject's oral cavity and swept through the oral cavity. This procedure is repeated three times a day during the duration of the hospital stay.

### EXAMPLE XIII

An adult male human subject without ventilator-associated pneumonia is prophylactically treated for ventilator-associated pneumonia during a hospital stay as follows. A lozenge containing antimicrobial, atomically disordered, nanocrystalline-silver containing material is prepared. The lozenge is inserted into the subject's oral cavity and sucked for 15 minutes. This procedure is repeated three times a day during the duration of the hospital stay.

### EXAMPLE XIV

An adult male human subject without ventilator-associated pneumonia is prophylactically treated for ventilator-associated pneumonia during a hospital stay as follows. A sponge containing antimicrobial, atomically disordered, nanocrystalline-silver containing material is prepared. The sponge is inserted into the subject's oral cavity and swept through the oral cavity. This procedure is repeated three times a day during the duration of the hospital stay.

### EXAMPLE XV

An adult male human subject without ventilator-associated pneumonia is prophylactically treated for ventilator-associated pneumonia during a hospital stay as follows. A foam article containing antimicrobial, atomically disordered, nanocrystalline-silver containing material is prepared. The foam article is inserted into the subject's oral cavity and swept through the oral cavity. This procedure is repeated three times a day during the duration of the hospital stay.

### EXAMPLE XVI

An adult male human subject without ventilator-associated pneumonia is prophylactically treated for ventilator-associated pneumonia during a hospital stay as follows. A tape containing antimicrobial, atomically disordered, nanocrystalline-silver containing material is prepared. The tape is inserted into the subject's oral cavity and kept in the oral cavity for 15 minutes. This procedure is repeated three times a day during the duration of the hospital stay.

### EXAMPLE XVII

An adult male human subject without ventilator-associated pneumonia is prophylactically treated for ventilator-associated pneumonia during a hospital stay as follows. A solution containing antimicrobial, atomically disordered, nanocrystalline-silver containing material is prepared. The subject rinses his oral cavity with the solution. This procedure is repeated three times a day during the duration of the hospital stay.

### EXAMPLE XVIII

An adult male human subject without ventilator-associated pneumonia is prophylactically treated for ventilator-associated pneumonia during a hospital stay as follows. A solution containing antimicrobial, atomically disordered, nanocrystalline-silver containing material is prepared. The subject gargles the solution. This procedure is repeated three times a day during the duration of the hospital stay.

### EXAMPLE XIX

An adult male human subject without ventilator-associated pneumonia is prophylactically treated for ventilator-associated pneumonia during a hospital stay as follows. A solution containing antimicrobial, atomically disordered, nanocrystalline-silver containing material is prepared. The solution is formed into a mist, and the subject inhales the mist through his oral cavity. This procedure is repeated three times a day during the duration of the hospital stay.

### EXAMPLE XX

An adult male human subject without ventilator-associated pneumonia is prophylactically treated for ventilator-associated pneumonia during a hospital stay as follows. A solution containing antimicrobial, atomically disordered, nanocrystalline-silver containing material is prepared. The solution is formed into a mist, and the subject inhales the mist through his nasal cavity. This procedure is repeated three times a day during the duration of the hospital stay.

### EXAMPLE XXI

An adult male human is operated on to remove a cancerous tumor. After removing the tumor, the affected area of the subject is sprayed with a solution containing antimicrobial, atomically disordered, nanocrystalline silver-containing material.

### EXAMPLE XXII

An adult male human subject is operated on to remove a cancerous skin lesion. After removing the lesion, the affected area of the subject is sprayed with a solution containing antimicrobial, atomically disordered, nanocrystalline silver-containing material.

### EXAMPLE XXIII

An adult male human is operated on to remove a cancerous tumor. After removing the tumor, the affected area of the subject is sprayed with a solution containing pro-apoptosis, atomically disordered, nanocrystalline silver-containing material.

### EXAMPLE XXIV

An adult male human subject is operated on to remove a cancerous skin lesion. After removing the lesion, the affected area of the subject is sprayed with a solution containing pro-apoptosis, atomically disordered, nanocrystalline silver-containing material.

### EXAMPLE XXV

An adult male human subject has a stent implanted in an artery to prevent restonosis. Prior to implantation, the stent is coated with antimicrobial, atomically disordered, nanocrystalline silver-containing material to prophylactically treat a microbial condition that could otherwise result from implantation of the stent.

### EXAMPLE XXVI

An adult male human has a condition that is treated by inhaling a drug in dry powder form with a dry powder inhaler. The subject is prophylactically treated for ventilator-associated pneumonia by contacting the dry powder inhaler with antimicrobial, atomically disordered, nanocrystalline silver-containing material before the inhaler is used by the subject.

### EXAMPLE XXVII

An adult male human has a condition that is treated by inhaling a drug in aerosol form with a mechanical mister. The subject is prophylactically treated for ventilator-associated pneumonia by contacting the mechanical mister with antimicrobial, atomically disordered, nanocrystalline silver-containing material before the mechanical mister is used by the subject.

### EXAMPLE XXVIII

An adult male human has a condition that is treated by inhaling a drug in dry powder form with a needleless injector. The subject is prophylactically treated for ventilator-associated pneumonia by contacting the needleless injector with antimicrobial, atomically disordered, nanocrystalline silver-containing material before the inhaler is used by the subject.

### EXAMPLE XXIX

An adult male is intubated to treat a respiratory condition. Prior to being intubated, the respiratory equipment is rinsed with antimicrobial, atomically disordered, nanocrystalline silver-containing material.

### EXAMPLE XXX

An adult male is intubated to treat a respiratory condition. Prior to being intubated, the respiratory equipment is washed with antimicrobial, atomically disordered, nanocrystalline silver-containing material.

### EXAMPLE XXXI

An adult male is intubated to treat a respiratory condition. Prior to being intubated, the respiratory equipment is coated with antimicrobial, atomically disordered, nanocrystalline silver-containing material.

Other embodiments are in the claims.

What is claimed is:

1. A method of prophylactically treating a condition, comprising:
contacting a first area of a subject with a metal-containing material to reduce the occurrence of the condition at a second area of the subject,
wherein the first area is different from the second area.

2. The method of claim 1, further comprising:
recognizing a possibility for occurrence of the condition at the second area of the subject; and
after, recognizing the possibility for occurrence of the condition at the second area of the subject, selecting the first area of the subject for contact with the metal-containing material to reduce occurrence of the condition at the second area of the subject.

3. The method of claim 1, wherein the second area is substantially free of the condition when the first area is contacted with the metal-containing material.

4. The method of claim 1, wherein the second area has the condition when the first area is contacted with the metal-containing material.

5. The method of claim 1, wherein the metal-containing material is selected from the group consisting of metals and alloys.

6. The method of claim 1, wherein the metal-containing material is selected from the group consisting of metal oxides, metal hydroxides, metal nitrides, metal borides, metal halides, metal carbides, metal phosphides, metal silicates, metal nitrates, metal carbonates, metal sulfides, metal sulfadiazines, metal acetates, metal lactates, metal citrates, metal myristates, metal sorbates, metal stearates, metal oleates, metal glutonates, metal adipates, alkali metal thiosulphates metal hydrides combinations thereof.

7. The method of claim 1, wherein the metal-containing material comprises a metal selected from the group consisting of silver, gold, platinum, palladium and combinations thereof

8. The method of claim 1, wherein the metal-containing material comprises silver.

9. The method of claim 1, wherein the metal-containing material comprises an ionic material.

10. The method of claim 1, wherein the metal-containing material comprises atoms, molecules or clusters.

11. The method of claim 1, wherein the metal-containing material comprises an atomically disordered, crystalline metal-containing material.

12. The method of claim 11, wherein the metal-containing material comprises a nanocrystalline metal-containing material.

13. The method of claim 12, wherein the metal-containing material comprises a material selected from the group consisting of antimicrobial metal-containing materials, anti-biofilm metal-containing materials, antibacterial metal-containing materials, anti-inflammatory metal-containing materials, antifungal metal-containing materials, antiviral metal-containing materials, anti-autoimmune metal-containing materials, anti-cancer metal-containing materials, pro-apoptosis metal-containing materials, anti-proliferative materials, MMP modulating metal-containing materials and combinations thereof.

14. The method of claim 11, wherein the metal-containing material comprises a material selected from the group consisting of antimicrobial metal-containing materials, anti-biofilm metal-containing materials, antibacterial metal-containing materials, anti-inflammatory metal-containing materials, antifungal metal-containing materials, antiviral metal-containing materials, anti-autoimmune metal-containing materials, anti-cancer metal-containing materials, pro-apoptosis metal-containing materials, anti-proliferative materials, MMP modulating metal-containing materials and combinations thereof.

15. The method of claim 1, wherein the metal-containing material comprises a nanocrystalline metal-containing material.

16. The method of claim 15, wherein the metal-containing material comprises a material selected from the group consisting of antimicrobial metal-containing materials, anti-biofilm metal-containing materials, antibacterial metal-containing materials, anti-inflammatory metal-containing materials, antifungal metal-containing materials, antiviral metal-containing materials, anti-autoimmune metal-containing materials, anti-cancer metal-containing materials, pro-apoptosis metal-containing materials, anti-proliferative materials, MMP modulating metal-containing materials and combinations thereof.

17. The method of claim 1, wherein the metal-containing material comprises a material selected from the group consisting of antimicrobial metal-containing materials, anti-biofilm metal-containing materials, antibacterial metal-containing materials, anti-inflammatory metal-containing materials, antifungal metal-containing materials, antiviral metal-containing materials, anti-autoimmune metal-containing materials, anti-cancer metal-containing materials, pro-apoptosis metal-containing materials, anti-proliferative, materials, MMP modulating metal-containing materials and combinations thereof.

18. The method of claim 1, wherein the condition is selected from the group consisting of bacterial conditions, biofilm conditions, microbial conditions, inflammatory conditions, fungal conditions, viral conditions, autoimmune conditions, hyperproliferative conditions, idiopathic conditions, cancerous conditions and combinations thereof.

19. The method of claim 1, wherein the condition is selected from skin conditions, integument conditions and combinations thereof.

20. The method of claim 19, wherein the condition is selected from the group consisting of bacterial conditions, biofilm conditions, microbial conditions, inflammatory conditions, fungal conditions, viral conditions, autoimmune conditions, idiopathic conditions, hyperproliferative conditions, cancerous conditions and combinations thereof.

21. The method of claim 19, wherein the condition is selected from the group consisting of a burn, eczema, erythroderma, an insect bite, mycosis fungoides, pyoderma gangrenosum, eythrema multiforme, rosacea, onychomycosis, acne, psoriasis, Reiter's syndrome, pityriasis rubra pilaris, hyperpigmentation, vitiligo, scarring conditions, keloid, lichen planus, age related skin disorders, hyperproliferative variants of the disorders of keratinization and combinations thereof.

22. The method of claim 1, wherein the condition comprises a respiratory condition.

23. The method of claim 22, wherein the condition is selected from the group consisting of bacterial conditions, biofilm conditions, microbial conditions, inflammatory conditions, fungal conditions, viral conditions, autoimmune conditions, idiopathic conditions, hyperproliferative conditions, cancerous conditions and combinations thereof.

24. The method of claim 22, wherein the respiratory condition is selected from. the group consisting of asthma, emphysema, bronchitis, pulmonary edema, acute respiratory distress syndrome, bronchopulmonary dysplasia, pulmonary fibrosis, pulmonary atelectasis, tuberculosis, pneumonia, sinusitis, allergic rhinitis, pharyngitis, mucositis, stomatitis, chronic obstructive pulmonary disease, bronchiectasis, lupus pneumonitis, cystic fibrosis and combinations thereof.

25. The method of claim 1, wherein the condition comprises a musculo-skeletal condition.

26. The method of claim 25, wherein the condition is selected from the group consisting of bacterial conditions, biofilm conditions, microbial conditions, inflammatory conditions, fungal conditions, viral conditions, autoimmune conditions, idiopathic conditions, hyperproliferative conditions, cancerous conditions and combinations thereof.

27. The method of claim 25, wherein the musculo-skeletal condition is selected from the group consisting of tendonitis, osteomyelitis, fibromyalgia, bursitis, arthritis and combinations thereof.

28. The method of claim 1, wherein the condition comprises a circulatory condition.

29. The method of claim 28, wherein the condition is selected from the group consisting of bacterial conditions, biofilm conditions, microbial conditions, inflammatory conditions, fungal conditions, viral conditions, autoimmune conditions, idiopathic conditions, hyperproliferative conditions, cancerous conditions and combinations thereof.

30. The method of claim 28, wherein the circulatory condition is selected from the group consisting of arteriosclerosis, lymphoma, septicemia, leukemia, ischemic vascular disease, lymphangitis, atherosclerosis and combinations thereof.

31. The method of claim 1, wherein the condition comprises cancer.

32. The method of claim 31, wherein the cancer is selected from the group consisting of tumors, hematologic malignancies and combinations thereof.

33. The method of claim 1, wherein the condition is selected from the group consisting of mucosal conditions, serosal conditions and combinations thereof.

34. The method of claim 33, wherein the condition is selected from the group consisting of bacterial conditions, biofilm conditions, microbial conditions, inflammatory conditions, fungal conditions, viral conditions, autoimmune conditions, idiopathic conditions, hyperproliferative conditions, cancerous conditions and combinations thereof.

35. The method of claim 33, wherein the condition is selected from the group consisting of pericarditis, Bowen's disease, stomatitis, prostatitis, sinusitis, allergic rhinitis, digestive disorders, peptic ulcers, esophageal ulcers, gastric ulcers, duodenal ulcers, toxic epidermal necrolysis syndrome, Stevens Johnson syndrome, cystic fibrosis, bronchitis, pneumonia, pharyngitis, common cold, ear infections, sore throat, sexually transmitted diseases, inflammatory bowel disease, colitis, hemorrhoids, thrush, dental conditions, oral conditions, conjunctivitis, periodontal conditions and combinations thereof.

36. The method of claim 1, wherein the first area of the subject is selected from the group consisting of a hyperplastic tissue, a tumor tissue, a cancerous lesion and combinations thereof.

37. The method of claim 1, wherein the second area of the subject is selected from the group consisting of a hyperplastic tissue, a tumor tissue, a cancerous lesion and combinations thereof.

38. The method of claim 1, wherein the method prophylactically induces apoptosis at the second area of the subject.

39. The method of claim 1, wherein the method prophylactically modulates matrix metalloproteinases at the second area of the subject.

40. The method of claim 1, wherein, when contacted with the subject, the metal-containing material is in a solution.

41. The method of claim 40, wherein the solution is injected.

42. The method of claim 41, wherein the solution is injected via a needleless injector.

43. The method of claim 41, wherein the solution is injected via a needle.

44. The method of claim 40, wherein the solution contains at least about 0.001 weight percent of the metal-containing material.

45. The method of claim 44, wherein the solution contains about 10 weight percent or less of the metal-containing material.

46. The method of claim 40, wherein the solution further comprises a solvent.

47. The method of claim 40, further comprising:
forming the solution into an aerosol; and
inhaling the aerosol.

48. The method of claim 1, wherein, when contacted with the subject, the metal-containing material is disposed in a pharmaceutically acceptable carrier.

49. The method of claim 48, wherein the composition contains at least about 0.01 weight percent of the metal-containing material.

50. The method of claim 49, wherein the composition contains about 50 weight percent or less of the metal-containing material.

51. The method of claim 48, wherein the pharmaceutically acceptable carrier is selected from the group consisting of creams, ointments, gels, sprays, solutions, drops, powders, lotions, pastes, foams, liposomes and combinations thereof.

52. The method of claim 1, wherein, when contacted with the subject, the metal-containing material is in the form of a free standing powder.

53. The method of claim 52, wherein the free standing powder is inhaled.

54. The method of claim 52, wherein the free standing powder is injected.

55. The method of claim 1, wherein the first area comprises a mucosal membrane and the second area comprises the subject's lungs.

56. The method of claim 55, wherein the mucosal membrane is selected from the group consisting of the subject's oral cavity and the subject's nasal cavity.

57. The method of claim 55, wherein the condition is nosocomial pneumonia or ventilator-associated pneumonia.

58. The method of claim 55, wherein the metal-containing material is in the form of a solution when contacted with the subject.

59. The method of claim 55, wherein the metal-containing material is in the form of a swab, a sponge, a foam, a liposome, a tape, a pill, a capsule, a tablet, a suppository or a lozenge when contacted with the subject.

60. The method of claim 1, wherein the first area is substantially free of the condition when the first contacted with the metal-containing material.

61. The method of claim 1, wherein the first area has the condition when the first contacted with the metal-containing material.

62. The method of claim 1, wherein the metal-containing material has a prophylactic ratio of about 0.95 or less for the condition.

63. The method of claim 1, wherein, when contacted with the first area of the subject, the metal-containing compound is not in the form of a dressing.

64. The method of claim 1, wherein the first area of the subject is not the subject's skin.

65. The method of claim 1, wherein the condition is not a bacterial condition.

66. The method of claim 1, wherein the metal-containing material is selected from the group consisting of silver nitrate, silver hydroxide, silver sulfadiazine, colloidal silver, silver carbonate, silver oxide, silver acetate, silver lactate, silver citrate, silver succinate, silver chlorate, silver sorbate, silver myristate, silver stearate, silver oleate, silver glutonate, silver adipate, alkali silver thiosulphate and combinations thereof.

67. A method of prophylactically treating pneumonia, comprising:
contacting an area of a subject with an atomically disordered, nanocrystalline silver-containing material to reduce the occurrence of pneumonia in the subject,
wherein the area of the subject is selected from the group consisting of the oral cavity and the nasal cavity.

68. The method of claim 67, wherein the condition is nosocomial pneumonia or ventilator-associated pneumonia.

69. The method of claim 67, wherein the metal-containing material is in the form of a solution when contacted with the subject.

70. The method of claim 67, wherein the metal-containing material is in the form of a swab, a sponge, a foam, a liposome, a tape, a pill, a capsule, a tablet, a suppository or a lozenge when contacted with the subject.

71. The method of claim 67, wherein the lungs of the subject are substantially free pneumonia when contacted with the metal-containing material.

72. The method of claim 67, wherein the metal-containing material has a prophylactic ratio of about 0.95 or less for pneumonia.

73. A method of prophylactically treating a condition, comprising:
contacting a first area of a subject with a metal-containing material to reduce the occurrence of the condition at a second area of the subject,
wherein the first area of the subject is an area of the subject other than the skin.

74. The method of claim 73, wherein the first and second areas of the subject are the same area of the subject.

75. The method of claim 73, wherein the first area of the subject is selected from the group consisting of a portion of the subject's respiratory system, a portion of the subject's musculo-skeletal system, a portion of the subject's circulatory system, a portion of the subject's gastrointestinal system, a portion of the subject's sublingual area, and a portion of the subject's subdermal area, a hyperplastic tissue and a tumor tissue.

76. A method of prophylactically treating a condition, comprising:
contacting a first area of a subject with a metal-containing material to reduce the occurrence of the condition at a second area of the subject,
wherein the metal-containing material is in a form other than a dressing.

77. The method of claim 76, wherein the first and second areas of the subject are the same area of the subject.

78. The method of claim 76, wherein the metal-containing material is in a form selected from the group consisting of a free standing powder, a solution, a pharmaceutically acceptable carrier and a powder impregnated material.

79. A method of prophylactically treating a condition, comprising:
contacting a first area of a subject with a metal-containing material to reduce the occurrence of the condition at a second area of the subject,
wherein the condition is a non-bacterial condition.

80. The method of claim 79, wherein the first and second areas of the subject are the same area of the subject.

81. The method of claim 79, wherein the condition is selected from the group consisting of bacterial conditions, biofilm conditions, microbial conditions, inflammatory conditions, fungal conditions, viral conditions, autoimmune conditions, hyperproliferative conditions, idiopathic conditions, cancerous conditions and combinations thereof.

82. A method of prophylactically treating a condition, comprising:
contacting an object with a metal-containing material to reduce the occurrence of the condition at an area of a subject,
wherein the object is intended to be contacted with the subject or a material in contact with the object is intended to be contacted with the subject.

83. The method of claim 82, further comprising:
recognizing a possibility for occurrence of the condition at the area of the subject; and
after, recognizing the possibility for occurrence of the condition at the area of the subject, selecting the object for contact with the metal-containing material to reduce occurrence of the condition at the area of the subject.

83. The method of claim 82, further comprising; after contacting the object with the metal-containing material, contacting the object with the subject.

84. The method of claim 83, wherein the object is contacted with the area of the subject.

85. The method of claim 83, wherein the object is contacted with a different area of the subject.

86. The method of claim 82, further comprising, after contacting the object with the metal containing material, transferring from the object to the subject the material intended to be transferred to the subject.

87. The method of claim 86, wherein the material transferred to the subject comprises a therapeutic agent.

88. The method of claim 86, wherein the material is transferred directly from the object to the subject.

89. The method of claim 86, wherein the material is contacted with the area of the subject.

90. The method of claim 86, wherein the object is contacted with a different area of the subject.

91. The method of claim 82, wherein the object is selected from the group consisting of medical devices, surgical instruments, catheters, respiratory equipment, mechanical misters, spray bottles, nebulizers, oxygen tents, dry powder inhalers, needles, needleless injectors, dressings, solution droppers, containers for a solution, and combinations thereof.

92. The method of claim 82, wherein the area of the subject is substantially free of the condition when the object is contacted with the metal-containing material.

93. The method of claim 82, wherein the area of the subject has the condition when the object is contacted with the metal-containing material.

94. The method of claim 82, wherein the metal-containing material is selected from the group consisting of metals and alloys.

95. The method of claim 82, wherein the metal-containing material comprises an atomically disordered, crystalline metal-containing material.

96. The method of claim 82, wherein the metal-containing material comprises a nanocrystalline metal-containing material.

97. The method of claim 82, wherein the metal-containing material comprises a material selected from the group consisting of antimicrobial metal-containing materials, anti-biofilm metal-containing materials, antibacterial metal-containing materials, anti-inflammatory metal-containing materials, antifungal metal-containing materials, antiviral metal-containing materials, anti-autoimmune metal-containing materials, anti-cancer metal-containing materials, pro-apoptosis metal-containing materials, anti-proliferative materials, MMP modulating metal-containing materials and combinations thereof.

98. The method of claim 82, wherein the condition is selected from the group consisting of bacterial conditions, biofilm conditions, microbial conditions, inflammatory conditions, fungal conditions, viral conditions, autoimmune conditions, hyperproliferative conditions, idiopathic conditions, cancerous conditions and combinations thereof.

99. The method of claim 82, wherein the area of the subject is selected from the group consisting of the oral cavity and the nasal cavity.

100. The method of claim 82, wherein the area of the subject is an area of the subject other than the skin.

101. The method of claim 82, wherein the object is in a form other than a dressing.

102. The method of claim 82, wherein the condition is a non-bacterial condition.
